(19) United States
(12) Patent Application Publication
(10) Pub. No.: US 2024/0013378 A1
(43) Pub. Date: Jan. 11, 2024

(54) Bone Level Measurements On Dental Images With Machine Learning Algorithms

(71) Applicants: Richard Ricci, New York, NY (US); Andrea Cambria, New York, NY (US)

(72) Inventors: Richard Ricci, New York, NY (US); Andrea Cambria, New York, NY (US)

(73) Assignees: Richard Ricci, New York, NY (US); Andrea Cambria, New York, NY (US)

(21) Appl. No.: 18/229,299

(22) Filed: Aug. 2, 2023

Related U.S. Application Data
(63) Continuation-in-part of application No. 16/683,116, filed on Nov. 13, 2019, now Pat. No. 10,748,650.

(51) Int. Cl. G06T 7/00; A61B 90/00; G16H 30/40; G16H 10/60

(52) U.S. Cl. CPC G06T 7/0012; A61B 90/37; G16H 30/40; G16H 10/60; G06T 2207/30008; G06T 2207/30036; G06T 2200/24; G06T 2207/20081

(57) ABSTRACT

Bone level measurements on dental images with machine learning algorithms is described. In an example scenario, a computer processor and memory receives a dental image. The dental image is processed with a heuristic periapical bitewing algorithm to classify the dental image. The dental image is then processed with a machine learning (ML) cementoenamel junction (CEJ) algorithm, a ML first intersection of coronal alveolar bone and tooth algorithm, a ML apex of root algorithm to produce an identified CEJ, an identified first intersection of coronal alveolar bone and tooth, an identified apex of root. The algorithm is configured to calculate the shortest contiguous distance from the identified CEJ to the identified first intersection of coronal alveolar bone and tooth (numerator) divided by the shortest contiguous distance from the identified CEJ to the identified apex of root (denominator) to produce a bone level percentage measurement which is associated with the dental image.

### CLAIM OF PRIORITY

This application claims priority to U.S. provisional application 62/875,319, filed on Jul. 17, 2019, for U.S. Pat. No. 10,748,650B1, which issued as U.S. Pat. No. 10,748,650 B1. The contents of said provisional application and U.S. Pat. No. 10,748,650 B1 are hereby incorporated by reference in their entirety

### FIELD OF THE EMBODIMENTS

The field of embodiments disclosed herein relates to a system or method utilizing machine learning algorithms for the measurement of bone levels on dental x-rays. The system processes dental x-rays obtained from users or providers using advanced machine learning algorithms. Wherein a dental x-ray is a dental image. The algorithms may be executed in any order and include a range of instructions for accurate bone level measurements. Some exemplary algorithm instructions for dental bone level measurements may include:

For the periapical dental image:
[1] Receive a dental image from a user or provider.
[2] Process the dental image with a heuristic periapical bitewing algorithm to classify a dental image as one of: a periapical (PA) dental image, a bitewing (BW) dental image, a non-dental image.
[3] Process the PA dental image with one or more machine learning (ML) algorithms, such as the ML Cementoenamel Junction (CEJ) algorithm, ML first intersection of coronal alveolar bone and tooth algorithm, and ML apex of root algorithm.
[4] Identify the CEJ on the PA dental image using the ML CEJ algorithm.
[5] Identify the first intersection of coronal alveolar bone and tooth on the PA dental image using the ML first intersection of coronal alveolar bone and tooth algorithm.
[6] Identify the apex of root on the PA dental image using the ML apex of root algorithm.
[7] Measure the shortest contiguous distance on the PA dental image from the identified CEJ to the identified first intersection of coronal alveolar bone and tooth.
[8] Measure the shortest contiguous distance on the PA dental image from the identified CEJ to the identified apex of root.
[9] Derive and calculate the bone level percentage measurement by dividing the measured shortest contiguous distance on the PA dental image from the identified CEJ to the identified first intersection of coronal alveolar bone (numerator) and tooth by the measured shortest contiguous distance on the PA dental image from the identified CEJ to the identified apex of root (denominator).

Identify a PA dental image that may be missing at least one of: the CEJ, the first intersection of coronal alveolar bone and tooth, the apex of root, and providing instructions to at least one of: a server, a processor, a microprocessor, a processing device to omit, process, or reclassify the calculation accordingly.

Identify a PA dental image with an obstructed view of at least one of: the CEJ, the first intersection of coronal alveolar bone and tooth, the apex of root, and providing instructions to at least one of: a server, a processor, a processing device to omit, process, or reclassify the calculation accordingly.

Associate the bone level percentage measurement with the PA dental image.

Insert the identified CEJ, the identified first intersection of coronal alveolar bone and tooth, the identified apex of root, and the bone level percentage measurement into the PA dental image or x-ray and provide to at least one of: a user, a provider, an e-commerce organization, a ML entity, an algorithm, a graphic user interphase (GUI).

Another exemplary algorithm instructions for dental bone level measurements may include:

For the BW dental image:
[1] Receive a dental image from a user or provider.
[2] Process the dental image with a heuristic periapical bitewing algorithm to classify a dental image as one of: a PA dental image, a BW dental image, a non-dental image.
[3] Process the BW dental image with one or more ML algorithms, such as the ML CEJ algorithm, ML first intersection of coronal alveolar bone and tooth algorithm.
[4] Identify the CEJ on the BW dental image using the ML CEJ algorithm.
[5] Identify the first intersection of coronal alveolar bone and tooth on the BW dental image using the ML first intersection of coronal alveolar bone and tooth algorithm.
[6] Measure the shortest contiguous distance on the BW dental image from the identified CEJ to the identified first intersection of coronal alveolar bone and tooth and process with an image aspect ratio algorithm to derive a bone level percentage or measurement.
[7] Identify a BW dental image that may be missing at least one of: the CEJ, the first intersection of coronal alveolar bone and tooth and providing instructions to at least one of: a server, a processor, a microprocessor, a processing device to omit, process, or reclassify the calculation accordingly.
[8] Identify a BW dental image with an obstructed view of at least one of: the CEJ, the first intersection of coronal alveolar bone and tooth and providing instructions to at least one of: a server, a processor, a microprocessor, a processing device to omit, process, or reclassify the calculation accordingly.
[9] Insert the identified CEJ, the identified first intersection of coronal alveolar bone and tooth and the bone level percentage or measurement into the BW dental image or x-ray and provide to at least one of: a user, a provider, an e-commerce organization, a ML entity, an algorithm, a graphic user interphase.

Exemplary algorithm instructions for dental bone level measurements of panoramic, cephalometric, and a non-dental images may include:

For a panoramic dental image:
[1] Process the panoramic dental image with at least one algorithm comprising: a ML CEJ algorithm, a ML first intersection of coronal alveolar bone and tooth algorithm, a ML apex of root algorithm.
[2] Identify a CEJ on the panoramic dental image with the ML CEJ algorithm.
[3] Identify a first intersection of coronal alveolar bone and tooth on the panoramic dental image with the ML first intersection of coronal alveolar bone and tooth algorithm.
[4] Identify an apex of root on a panoramic dental image with the ML apex of root algorithm.
[5] Measure the shortest contiguous distance on a panoramic dental image from the identified CEJ to the identified first intersection of coronal alveolar bone and tooth.

[6] Measure the shortest contiguous distance on the panoramic dental image from the identified CEJ to the identified apex of root.
[7] Calculate and derive the measured shortest contiguous distance on the PA dental image from the identified CEJ to the identified first intersection of coronal alveolar bone and tooth (numerator) divided by the measured shortest contiguous distance on the PA dental image from the identified CEJ to the identified apex of root (denominator) to produce a bone level percentage measurement.
[8] Associate the bone level percentage measurement with the panoramic dental image.

For a cephalometric dental image:
[1] Process the cephalometric dental image with at least one algorithm comprising: a ML CEJ algorithm, a ML first intersection of coronal alveolar bone and tooth algorithm, a ML apex of root algorithm.
[2] Identify a CEJ on the cephalometric dental image with the ML CEJ algorithm.
[3] Identify a first intersection of coronal alveolar bone and tooth on the cephalometric dental image with the ML first intersection of coronal alveolar bone and tooth algorithm.
[4] Identify an apex of root on a cephalometric dental image with the ML apex of root algorithm.
[5] Measure the shortest contiguous distance on a cephalometric dental image from the identified CEJ to the identified first intersection of coronal alveolar bone and tooth.
[6] Measure the shortest contiguous distance on the cephalometric dental image from the identified CEJ to the identified apex of root.
[7] Calculate and derive the measured shortest contiguous distance on the PA dental image from the identified CEJ to the identified first intersection of coronal alveolar bone and tooth (numerator) divided by the measured shortest contiguous distance on the PA dental image from the identified CEJ to the identified apex of root (denominator) to produce a bone level percentage measurement.
[8] Associate the bone level percentage measurement with the cephalometric dental image.

For a non-dental image:

A non-dental image is replaced with a notification that is provided to at least one of: a user, a provider, an e-commerce organization, a ML entity, an algorithm. Further at least one of: a server, a processor, a microprocessor, a processing device is configured to continue processing the next image after a non-dental image is replaced with a notification.

These embodiments enable accurate and efficient bone level measurements on dental images. Thus, facilitating improved diagnosis and treatment planning.

### BACKGROUND OF THE EMBODIMENTS

Digital dental images have revolutionized the field of dentistry, bringing about significant advancements in patient care and treatment. With the widespread adoption of digital radiography, dental offices worldwide have embraced the benefits of this technology. Today, dentists, hygienists, and dental staff are extensively trained in capturing and processing digital dental images, making them an integral part of routine dental procedures. Compared to traditional film dental x-rays, digital dental x-rays offer numerous advantages. Firstly, digital images can be processed at a much faster rate, allowing dental professionals to swiftly review and analyze patient data. This enhanced efficiency translates into improved productivity and reduced waiting times for patients. Furthermore, digital radiography significantly reduces a patient's radiation exposure, promoting a safer and more comfortable experience during dental procedures.

The advent of digital dental imaging has paved the way for comprehensive patient dental image management services. These services encompass a wide range of applications, such as offsite image hosting, seamless integration of dental images with insurance claims, dental laboratory scans, x-ray to graphic-based charting, and even dental charting by voice command. By leveraging these advanced image management capabilities, dental practices can streamline their workflows, enhance accuracy, and provide better overall patient care.

While existing dental x-ray platforms often feature manual millimeter measurement tools displayed in their GUI, none have incorporated a ML or artificial intelligence percentage measurement algorithm tool into their systems. This presents an unmet need within the industry. It should be understood that a manual millimeter measurement tools are prone to human error. By developing a dental image platform equipped with a percentage measurement algorithm, dental professionals would gain a valuable tool for assessing bone level loss. This would enable dentists and hygienists to identify and diagnose marginal to moderate periodontal disease more effectively, which is often underdiagnosed using conventional methods. In addition to diagnosis, a ML or artificial intelligence percentage measurement algorithm tool would empower dental practitioners to monitor the progression of a patient's periodontal disease over time. By tracking changes in bone level measurements, clinicians can tailor treatment plans and interventions accordingly. Hence promoting proactive management and better treatment outcomes for patients.

Moreover, the incorporation of a ML or artificial intelligence percentage measurement algorithm tool into dental image platforms would also have a profound impact on patient engagement and education. Patients who have a clearer understanding of their periodontal conditions are more likely to actively participate in their own oral health management. By visualizing and comprehending the percentage based bone level measurements, patients can make informed decisions about their treatment options and take necessary steps towards early intervention. This, in turn, contributes to an elevated standard of dental care and improved long-term oral health outcomes.

Given the undeniable societal and professional demand for this technological advancement, there is a pressing need for the development and implementation of a dental image platform that incorporates an AI percentage measurement algorithm tool. This innovation would not only bridge a current gap in the field but also enhance the quality of care delivered to patients. Thus leading to better oral health outcomes and improved overall well-being.

### SUMMARY OF THE EMBODIMENTS

The present invention and its embodiments relates to bone level measurements on dental images with ML algorithms. The system or method may include may use ML algorithms to calculate bone level measurements on dental images. The system or method may include may include at least one of: at least one of: a server, a processor, a microprocessor, a processing device. At least one of: a server, a processor, a microprocessor, a processing device may be configured to receive a dental image of a patient from a user, a provider, an e-commerce organization, a ML entity, an algorithm. An example of a user or provider may include a patient, a dentist, a doctor, an insurance company, a bioinformatics organization, a business, an e-commerce organization, a ML entity, an algorithm, a cloud based storage service, among others. At least one of: a server, a processor, a microprocessor, a processing device may be configured to differentiate between a PA dental image and a BW dental image with a heuristic periapical bitewing algorithm. The dental image may be processed with the heuristic periapical bitewing algorithm to classify a dental image as one of: a PA dental image, a BW dental image. PA dental images may be used to calculate bone level measurements. Next, the PA dental image may be processed with a ML CEJ algorithm. The PA dental image may also be processed with a ML first intersection of coronal alveolar bone and tooth algorithm and a ML apex of root algorithm. The algorithms configured to measure the shortest contiguous distance on the PA dental image from the identified CEJ to the identified first intersection of coronal alveolar bone and tooth and then measure the shortest contiguous distance on the PA dental image from the identified CEJ to the identified apex of root. The algorithm is further configured to process and calculate: the measured shortest contiguous distance on the PA dental image from the identified CEJ to the identified first intersection of coronal alveolar bone and tooth and the measured shortest contiguous distance on the PA dental image from the identified CEJ to the identified apex of root to derive a bone level percentage measurement. The algorithm may associate the bone level percentage measurement with the PA dental image as at least one of: a percentage, a quotient, a ratio, a whole number, a proportion, an average, a median, a standard deviation. The measurement and the PA dental image may be a displayed on a user interface such as a GUI.

The system or method of the embodiment may utilize components such as one or more of: a server, a processor, a microprocessor, a processing device and may be further configured to process the PA dental image with an apex of an infrabony pocket algorithm. This algorithm may process the PA dental image with at least one of: a ML CEJ algorithm, a ML apex of infrabony pocket algorithm, a ML apex of root algorithm. Match and identify a CEJ on the PA dental image with the ML CEJ algorithm to produce an identified CEJ. Match and identify an apex of an infrabony pocket on the PA dental image with the ML apex of infrabony pocket algorithm to produce an identified apex of an infrabony pocket. Wherein an infrabony pocket is the first intersection of coronal alveolar bone and tooth. Wherein an infrabony pocket is also known as: an intrabony periodontal pocket, a subcrestal pocket, and an intraalveolar pocket. Match and identify an apex of root on the PA dental image with a ML apex of root algorithm to produce an identified apex of root. Identify and compensate for a PA dental image missing at least one of: a CEJ, an apex of infrabony pocket, an apex of root and instruct the processor to at least one of: omit, process, reclassify the PA dental image. Identify and compensate a PA dental image with an obstructed view of at least one of: a CEJ, an apex of infrabony pocket, an apex of root and instruct the processor to at least one of: omit, process, reclassify the PA dental image. Wherein at least one of: a system, a processor, a microprocessor, a processing device is configured to compensate for least one of: a CEJ, a ML first intersection of alveolar bone, a tooth apex of root that is missing or obstructed. Measure a distance on the PA dental image from the identified CEJ to the identified apex of an infrabony pocket and measure a distance on the PA dental image from the identified CEJ to the identified apex of root. Process and calculate the distance from the identified CEJ to the identified apex of an infrabony pocket (numerator) and divide by the distance from the identified CEJ to the identified apex of root (denominator) to produce a bone level percentage measurement. Associate the bone level percentage measurement with the PA dental image. The algorithm or processor may query and receive a patient data and correlate the patient data with the PA dental image and provide to the correlation dataset. It should be understood that a numerator may also be referred to as dividend and a denominator may also be referred to as a divisor. Further, at least one of: a server, a processor, a microprocessor, a processing device may be configured to compensate for a location variance of at least one of: the identified CEJ, the identified first intersection of coronal bone and tooth, the identified apex of an infrabony pocket, the identified tooth apex. The algorithm may format the dental image based on a transaction processed by at least one of: a user, a provider, an e-commerce organization, a ML entity, an algorithm; merge the dental image with a transaction processed by at least one of: a user, a provider, an e-commerce organization, a ML entity, an algorithm into a correlation dataset and identify and correct a discrepancy between the dental image with a transaction processed by at least one of: a user, a provider, an e-commerce organization, a ML entity, an algorithm, and the correlation dataset.

In another embodiment of the present invention the system or method may include may include at least one of: a server, a processor, a microprocessor, a processing device for providing bone level measurements on dental images with ML algorithms is described. At least one of: a server, a processor, a microprocessor, a processing device may include a computer vision component configured to analyze the dental image, a memory configured to store instructions associated with at least one of: a server, a processor, a microprocessor, a processing device that may be coupled with a computer vision component and the memory. At least one of: a server, a processor, a microprocessor, a processing device may execute the instructions associated with a ML algorithm. At least one of: a server, a processor, a microprocessor, a processing device may process an instruction in any order. At least one of: a server, a processor, a microprocessor, a processing device may include image processing algorithms or an image processing engine. The image processing algorithms or the image processing engine may be configured to receive a dental image of a patient from at least one of: a user, a provider. An example of a dental image user or provider may include a patient, a dentist, a doctor, an insurance company, a bioinformatics organization, a business, an e-commerce organization, a ML entity, an algorithm, a cloud based storage service, among others.

In another embodiment, the system or method may provide an anatomic delineation percentage measurement of a dental image. The system or method may comprise at least one of: a server, a processor, a processing device that may be configured to: receive a dental image, may execute an instruction in any order, process the dental image using at least one ML algorithm, comprising: a ML anatomy algorithm and/or a ML pathology algorithm to identify anatomic delineations in the dental image, measure the distance on the dental image between a first identified anatomic delineation and a second identified anatomic delineation, and the distance on the dental image between a first identified anatomic delineation and a third identified anatomic delineation. Calculate the anatomic delineation percentage measurement by dividing the distance on the dental image between the first and second identified anatomic delineations (numerator) by the distance on the dental image between the first and third identified anatomic delineations (denominator) and associate the anatomic delineations percentage measurement with the dental image. In addition, a patient data or dataset of the patient associated with the dental image may be queried and received from a patient data user or provider and then associated with the dental image. It should be understood that an initially provided dental x-ray associated with a patient data shall be omitted from processing. One or more of: a dental image, a first identified anatomic delineation, a second identified anatomic delineation, a third identified anatomic delineation, an anatomic delineations percentage measurement may be inserted to the patient dataset. Furthermore, a cluster analysis of the patient dataset may be performed with a cluster dataset or an algorithm to produce a correlation dataset or a correlated dental information. Moreover the correlation dataset or the correlated dental information may be provided to a user, a provider, an e-commerce organization, a ML entity, an algorithm. A user or a provider may also use the correlation dataset or the correlated information as a diagnostic aid which may be provided to a GUI.

In yet another embodiment of the present invention the system or method for providing bone level measurements on dental images with ML algorithms is described. The system or method may include may include receiving a dental image of a patient from a dental image provider. An example of the dental image provider may include: a patient, a dentist, a doctor, an insurance organization, a bioinformatics organization, an e-commerce service, a ML entity, an algorithm, a cloud based storage service, among others. The dental image may next be processed with at least one of: a server, a processor, a microprocessor, a processing device. The said microprocessor may be configured to: executing an instruction in any order, receiving said dental image of a patient from a dental image provider, processing said dental image with at least one of: a heuristic periapical bitewing algorithm to classify a dental image as one of: a PA dental image, a BW dental image. At least one of: a server, a processor, a microprocessor, a processing device may be configured for processing the dental image with one or more of: a ML CEJ algorithm, a ML first intersection of coronal alveolar bone and tooth algorithm, a ML apex of root algorithm. Identifying a CEJ on said PA dental image with said ML CEJ algorithm to produce an identified CEJ. Identifying a first intersection of coronal alveolar bone and tooth on said PA dental image with said first intersection of alveolar bone and tooth algorithm to produce an identified first intersection of coronal alveolar bone and tooth. Identifying an apex of root on said PA dental image with said ML apex of root algorithm to produce an identified apex of root. Measuring the shortest contiguous distance on said PA dental image from said identified CEJ to said identified first intersection of coronal alveolar bone and tooth and measuring the shortest contiguous distance on said PA dental image from said identified CEJ to said identified apex of root. Processing and calculating to at least one of: derive a percentage, derive a quantitative score on said PA dental image said measured shortest contiguous distance on the PA dental image from said identified CEJ to said identified first intersection of alveolar bone and tooth (numerator) and divide by said measured shortest contiguous distance on the PA dental image from said identified CEJ to said identified apex of root (denominator) to produce a bone level percentage measurement. Processing said BW dental image with at least one algorithm comprising: said ML CEJ algorithm, said ML first intersection of coronal alveolar bone and tooth algorithm. Identifying a CEJ on said BW dental image with said ML CEJ algorithm to produce an identified CEJ. Identify a first intersection of coronal alveolar bone and tooth on said BW dental image with said ML first intersection of coronal alveolar bone and tooth algorithm to produce an identified first intersection of coronal alveolar bone and tooth. Measuring the shortest contiguous distance on said BW dental image from said identified CEJ to said identified first intersection of coronal alveolar bone and tooth algorithm and process with an image aspect ratio algorithm to produce a bone level percentage measurement, a measurement. Wherein a bone level percentage measurement and/or measurement may be a quantitative score. Identifying said PA or BW dental image missing at least one of: a CEJ, a first intersection of coronal alveolar bone and tooth, an apex of root and instruct to at least one of: omit, process, reclassify said calculation. Identifying said PA or BW dental image with a distorted view of at least one of: a CEJ, a first intersection of coronal alveolar bone and tooth, an apex of root and instruct to at least one of: omit, process, reclassify said calculation. Querying and receiving a patient data of said patient associated and with said PA or BW dental image. Associating said PA or BW dental image and said patient data with at least one of: said identified CEJ, said identified first intersection of coronal alveolar bone and tooth, said identified apex of root, said shortest contiguous distance on said PA dental image from said identified CEJ to said identified first intersection of coronal alveolar bone and tooth, said shortest contiguous distance on said PA dental image from said identified CEJ to said identified apex of root, said bone level percentage measurement, said shortest contiguous distance on said BW dental image from said identified CEJ to said identified first intersection of coronal alveolar bone and tooth, said bone level percentage measurement, said measurement, said patient data to produce a correlated dental information or correlation dataset. Providing said correlated dental information and/or correlation dataset to a memory. Providing correlated dental information and/or correlation dataset to one or more of: a user, a provider, an e-commerce organization, an insurance company, a bioinformatics organization, a business, a ML entity, a cloud based storage, an algorithm.

At least one of: a server, a processor, a microprocessor, a processing device may be configured to compensate for at least one of: a distorted image information, a missing image information, an obstructed image information. At least one of: a server, a processor, a microprocessor, a processing device may identify said PA or BW dental image missing at least one of: a CEJ, a first intersection of coronal alveolar bone and tooth, an apex of root and instruct to at least one of: omit, process, reclassify said calculation. Identify said PA or BW dental image with a distorted view of at least one of: a CEJ, a first intersection of coronal alveolar bone and tooth, an apex of root and instruct to at least one of: omit, process, reclassify said calculation. It should be understood, that a server, a processor, a processing device may also be configured to compensate for at least one of: a distorted image information, a missing image information, an obstructed image information on said PA or BW dental image.

Furthermore, a cluster analysis of the correlated dental information or a correlation dataset may be processed with a cluster dataset to produce additional information. Moreover, the correlated dental information or correlation dataset may be provided to an e-commerce company, an insurance company, a bioinformatics company, a business, a ML company, a cloud based company. The correlated dental information or correlation dataset may also be provided to a user or provider as a diagnostic aid.

Processing a dental image with a non-described algorithm and/or a non-described data may disrupt the invention's ability to process bone level measurements on dental images with ML algorithms. The invention will be programed to omit non-described algorithms and/or non-described data associated with a dental image. Wherein at least one of: a server, a processor, a microprocessor, a processing device may replace at least one of: a non-described algorithm, a non-described data with at least of: an algorithm, a data and continue processing. Wherein at least one of: a server, a processor, a microprocessor, a processing device will be programed to omit non-described algorithm associated with a dental image and replace with a new algorithm and continue processing. Wherein at least one of: a server, a processor, a microprocessor, a processing device will be programed to omit non-described data associated with a dental image and replace with a new data and continue processing. The invention may replace a non-described algorithm with a data and continue processing. The invention may replace a non-described data with an algorithm and continue processing. At least one of: a server, a processor, a microprocessor, a processing device is configured to omit processing a dental image with at least one of: a non-described algorithm, a non-described data. Wherein a non-described algorithm or a non-described data may include one or more of: a dental image set or image set with a label, object sub-types, confidence scores, a probability value of an image class, an image class vector, an image class space, a field of view label, a shallow hash neural network, a hash neural network, laboratory records of a patient, a laboratory test data, unified formats of lab test data, lab data off different formats, a computer code, a computer data. Wherein a lab is a laboratory. Wherein a label identifies a region of a particular anatomic structure. At least one of: a server, a processor, a microprocessor, a processing device may replace the omit processing with another data or algorithm and continuing processing. At least one of: a server, a processor, a microprocessor, a processing device may identify a discrepancy is at least one of: a non-described algorithm, a non-described data. Further, at least one of: a server, a processor, a microprocessor, a processing device is configured to omit processing on an augmented reality display and replaced with a GUI. Further, at least one of: a server, a processor, a microprocessor, a processing device is configured to omit processing a dental image with a non-described algorithm and/or a non-described data; wherein a non-described algorithm and/or non-described data at least one of: a dental image landmark probabilities dataset, an image class landmark probabilities dataset, an object class landmark probabilities dataset, a spatial landmark probability relationships dataset, an object probability landmarks dataset, an object probability relationships dataset, a dental image landmark probability map, a dental image landmark probability map dataset, a dental image dataset, a dataset, a computer code, a computer data and may replace the omit processing with another algorithm and continue processing.

Described algorithms include: a heuristic periapical bitewing algorithm, a ML CEJ algorithm, a ML first intersection of coronal alveolar bone and tooth algorithm, a ML apex of root algorithm, an apex of infrabony pocket algorithm, a ML anatomy algorithm, a ML pathology algorithm. Described data include: an identified CEJ, the identified first intersection of coronal alveolar bone and tooth, an identified apex of root, the shortest contiguous distance on the PA dental image from the identified CEJ to the identified first intersection of coronal alveolar bone and tooth, the shortest contiguous distance on the PA dental image from the identified CEJ to the identified apex of root, a bone level percentage measurement, a bone level percentage or measurement, a formatted bone level percentage or measurement, a first identified anatomic delineation, a second identified anatomic delineation, a third identified anatomic delineation, an anatomic delineation percentage or measurement, a missing CEJ, a missing the first intersection of coronal alveolar bone and tooth, a missing apex of root, an obstructed view of CEJ, an obstructed view of the first intersection of coronal alveolar bone and tooth, an obstructed view of the apex of root, a correlated dental information, a correlation dataset.

It is an object of the embodiments of the present invention to provide bone level measurements on dental images with ML algorithms.

It is an object of the embodiments of the present invention to determine, using a heuristic periapical bitewing algorithm, to classify a dental image as one of: a PA dental image, a BW dental image, a panoramic dental image, a cephalometric dental image, a non-dental image. Wherein a non-dental image is replaced with a notification that is provided to at least one of: a user, a provider, an e-commerce organization, a ML entity, an algorithm and the algorithm continues processing the next image.

It is an object of the embodiments of the present invention to determine a location of a CEJ, a first intersection of coronal alveolar bone and tooth, an apex of root associated on a PA dental image.

It is an object of the embodiments of the present invention to determine a location of a CEJ, a first intersection of coronal alveolar bone and tooth associated on a BW dental image.

It is an object of the embodiments of the present invention to calculate the measured shortest contiguous distance on the PA dental image from the identified CEJ to the identified first intersection of coronal alveolar bone and tooth and the measured shortest contiguous distance on the PA dental image from the identified CEJ to the identified apex of root to derive the percentage of bone loss on a PA dental image. A bone level percentage may be expressed as at least one of: decimal, quotient, percentage, a ratio, a whole number, a proportion, an average, a median, a standard deviation.

It is an object of the embodiments of the present invention to calculate the measured shortest contiguous distance on the BW dental image from the identified CEJ to the identified first intersection of coronal alveolar bone and tooth and process with an image aspect ratio algorithm to derive a bone level percentage or measurement on a BW dental image. A bone level percentage or measurement may be expressed as at least one of: decimal, quotient, percentage, a ratio, a whole number, a proportion, an average, a median, a standard deviation.

It is an object of the embodiments of the present invention to be configured for displaying at least one of: the identified CEJ, the identified first intersection of coronal alveolar bone and tooth, an identified apex of root, the shortest contiguous distance on the PA dental image from the identified CEJ to the identified first intersection of coronal alveolar bone and tooth, the shortest contiguous distance on the PA dental image from the identified CEJ to the identified apex of root, the bone level measurement, the bone level percentage or measurement, a first identified anatomic delineation, a second identified anatomic delineation, a third identified anatomic delineation, an anatomic delineation percentage measurement, the PA dental image, the BW dental image, the patient data, a correlated dental information, correlation dataset on a user interface.

It is an object of the embodiments of the present invention to associate a patient data with at least one of: the identified CEJ, the identified first intersection of coronal alveolar bone and tooth, an identified apex of root, the shortest contiguous distance on the PA dental image from the identified CEJ to the identified first intersection of coronal alveolar bone and tooth, the shortest contiguous distance on the PA dental image from the identified CEJ to the identified apex of root, the bone level measurement, the bone level percentage or measurement, a first identified anatomic delineation, a second identified anatomic delineation, a third identified anatomic delineation, an anatomic delineation percentage measurement, the PA dental image, the BW dental image, the patient data, a correlated dental information, correlation dataset and produce a diagnostic aid for a user or provider.

These and other features, aspects and advantages of the present invention will become better understood with reference to the following drawings, descriptions and claims.

### BRIEF DESCRIPTION OF THE DRAWINGS

FIG. 1 shows a display diagram illustrating examples of dental anatomy according to an embodiment of the invention.

FIG. 2 shows a display diagram illustrating examples of anatomic measurements of a tooth according to an embodiment of the invention.

FIG. 3 shows a display diagram illustrating a mesial, a distal, a buccal, a lingual, a facial surface of a tooth according to an embodiment of the invention.

FIG. 4 shows a display diagram illustrating difference techniques in capturing PA dental x-rays verses BW dental x-rays according to an embodiment of the invention.

FIG. 5 is a block diagram of an example computing device, which may include at least one of: a server, a processor, a microprocessor, a processing device which may be used to provide a bone level measurement on dental images with ML algorithms according to an embodiment of the invention.

### DESCRIPTION OF THE PREFERRED EMBODIMENTS

The preferred embodiments of the present invention will now be described with reference to the drawings. Examples of ML of dental images for bone level measurements on dental images with ML algorithms are discussed in this section. Identical elements in the various figures are identified with the same reference numerals.

Reference will now be made in detail to each embodiment of the present invention. Such embodiments are provided by way of explanation of the present invention, which are not intended to be limited thereto. In fact, those of ordinary skill in the art may appreciate upon reading the present specification and viewing the present drawings that various modifications and variations may be made thereto.

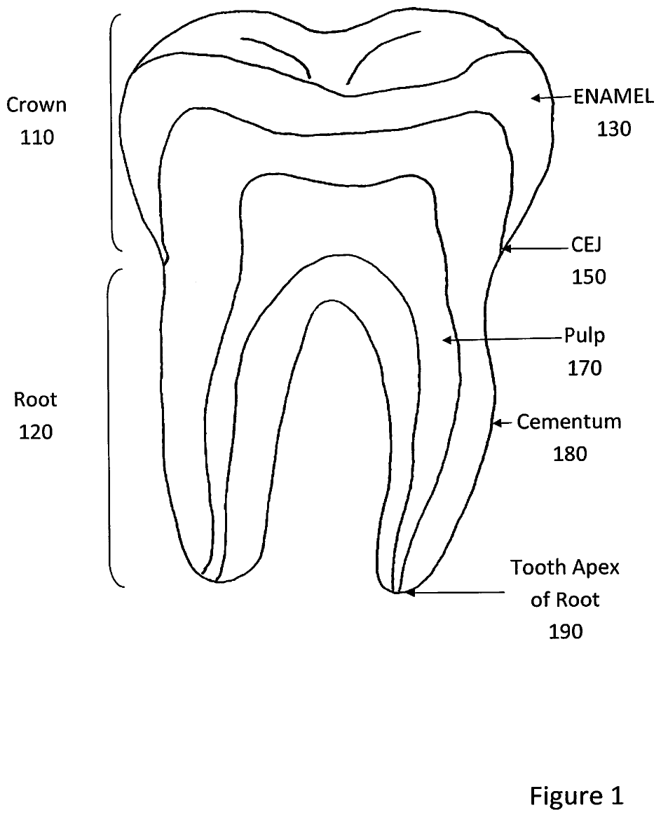

FIG. 1 shows a conceptual diagram illustrating basic human tooth anatomy. The human tooth is composed of numerous anatomical structures such as the crown 110, root 120, enamel 130, dentin 140, cementoenamel junction (CEJ) 150, pulp 170, cementum 180 and apex of root 190. Anatomic definitions of crown 110, root 120, enamel 130, dentin 140, cementoenamel junction (CEJ) 150, pulp 170, cementum 180, apex of root 190 in this document may be referenced in Woelfel, J. B. (1984), Dental Anatomy, Philadelphia: Lea & Febiger. Human teeth also have numerous associated anatomic structures such as the alveolar bone, the alveolar crest, infrabony pockets, periosteum, and periodontal ligaments.

In FIG. 1 the CEJ 150 is shown as the point where the enamel 130 of the tooth ends and the cementum 180 of the tooth begins. On a digital dental x-ray the CEJ 150 appears as the last white pixel or small grouping of pixels of enamel 130 before the cementum 180 which appear as gray pixels begins. It should be understood that the CEJ 150 may be a single point on a digital x-ray or may be a few scattered pixels with a high likelihood that the exact CEJ 150 pixel is within these few scattered pixels. The embodiment of this invention will utilize the CEJ 150 as an anatomic marker. A standard size 2 dental x-ray is 800 pixels by 640 pixels. Whether the CEJ 150 is an exact pixel or a few scattered pixels it will have a negligible effect on the calculation of bone level measurements.

A second aspect of the invention will utilize the first intersection of coronal alveolar bone and tooth 160 surface as an anatomic marker. This may also be referred to as the apex of the infrabony pocket. Further, this point is normally adjacent to the root 120 surface of the tooth. The exception to this are retained or impacted teeth. It should be understood that the first intersection of coronal alveolar bone and tooth 160 may be a single point on a digital x-ray or may be a few scattered pixels with a high likelihood that the exact first intersection of coronal alveolar bone and tooth 160 is within these few scattered pixels. A standard size 2 dental x-ray is 800 pixels by 640 pixels. Whether the first intersection of coronal alveolar bone and tooth 160 is an exact pixel or a few scattered pixels it will have a negligible effect in the calculation of bone level measurements.

A third aspect of the invention is the tooth's apex of root, which is the lowest point on the root 120 surface. It appears as a white pixel or group of pixels on a parabolic outline on the apex of the root. The third aspect of this invention will utilize the apex of root as an anatomic marker. Typically, the nerve of the tooth exits through a foramen in the apex of root. The nerve proceeds to connect to a branch of the either the maxillary V₂ or mandibular V₃ division of the trigeminal nerve. Apex of roots may have slight anatomic variances. Some may be curved, tilted or overlapped with another root 120. On a digital dental x-ray, the exact pixel of a tooth's apex of root might be challenging to identify. The apex of root may be a single point on a digital x-ray or may be a few scattered pixels on the lowest apical point of a parabolic outline. It should be understood that the apex of root may be a single point on a digital x-ray or may be a few scattered pixels with a high likelihood that the exact apex of root pixel is within these few scattered pixels. Whether the apex of root is an exact pixel or a few scattered pixels it will have a negligible effect in the calculation of bone level measurements. Further, wherein at least one of: a CEJ, a first intersection of coronal alveolar bone and tooth, an apex of root may be at least one of: a single pixel, several scattered pixels.

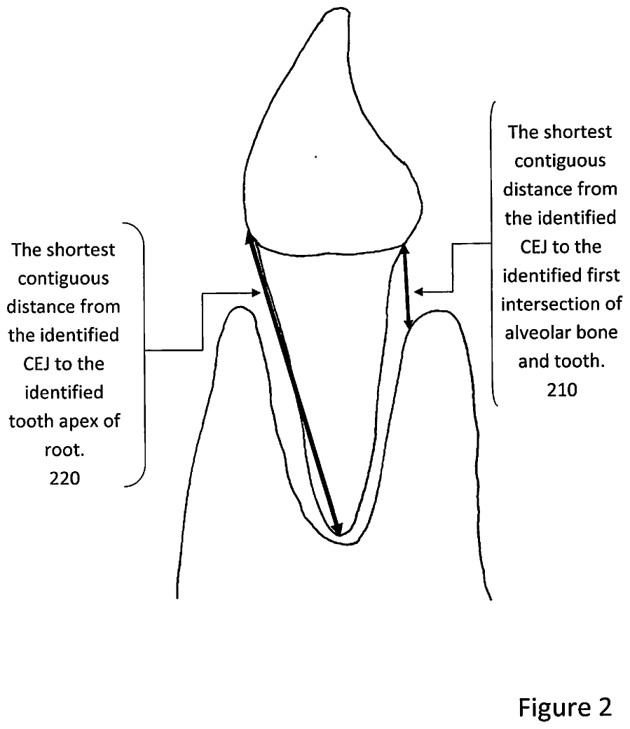

FIG. 2 shows two key measurements which is the forth aspect of the invention. The shortest contiguous distance on a PA dental image from the CEJ to the first intersection of coronal alveolar bone and tooth 210 and the shortest contiguous distance on the PA dental image from the CEJ to an apex of root 220. The invention is further configured to compensate for a location variance of at least one of: a CEJ 150, a first intersection of coronal alveolar bone and tooth 160, an apex of root 190.

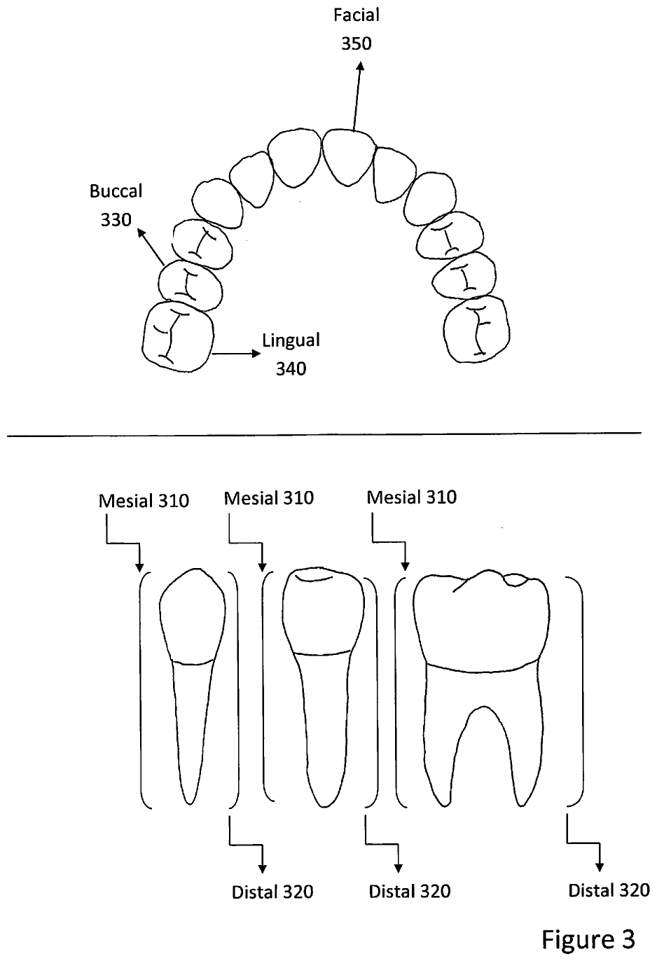

The dental field has created specific nomenclatures to describe specific anatomic locations on and around teeth. FIG. 3 displays these anatomic locations. These nomenclatures are different than are in the medical field. These nomenclatures enable dental professionals to effectively communicate spatial relationships on dental x-rays and dental images. Examples of these nomenclatures include: mesial 310, distal 320, buccal 330, lingual 340 and facial 350. To further clarify any PA dental image calculation deriving a percentage of bone level measurement of the measured shortest contiguous distance on the PA dental image from the CEJ to the first intersection of coronal alveolar bone and tooth 210 and the shortest contiguous distance on the PA dental image from the CEJ to the apex of root must both be located on the same side of the tooth. Any BW dental image calculation deriving a percentage of bone level measurement of the measured shortest contiguous distance on the BW dental image from the CEJ to the first intersection of coronal alveolar bone and tooth 210 must also be on the same side of the tooth. A side being one of: a mesial 310, a distal 320, a buccal 330, a lingual 340, a facial 350 surface of the tooth.

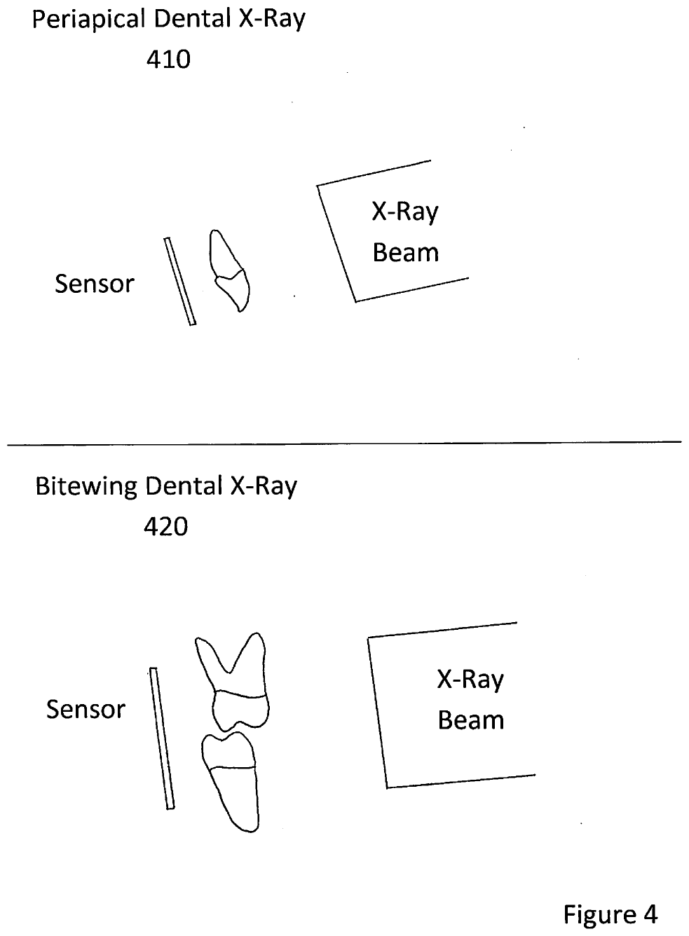

FIG. 4 shows what a PA dental x-ray 410 and what a BW dental x-ray 420 looks like. FIG. 4 further shows how a sensor is positions for obtaining a PA or BW dental x-ray. It should be understood that a dental x-ray is also known as a dental image. A PA is a type of radiographic image that provides a detailed view of an entire tooth and its surrounding structures. It is primarily used in dentistry to evaluate the condition of the tooth, root, the supporting bone, and the surrounding tissues. The term “periapical” refers to the area around the tooth. Therefore, a PA dental x-ray 410 focuses on capturing the whole tooth, from the crown to the root, along with the surrounding bone and tissues. This type of x-ray is commonly used to diagnose various dental conditions such as dental caries (cavities), periodontal diseases, abscesses, cysts, impacted teeth, infrabony pockets and other abnormalities that may affect the tooth, root or its adjacent structures. In comparison a BW dental x-ray 420 is a type of radiographic image used in dentistry to provide a detailed view of the upper and lower teeth in a posterior region of the mouth. The term “bitewing” refers to the method of capturing the x-ray, where the patient bites down on a tab or wing-shaped device that holds the digital sensor in place. BW dental x-rays 420 are primarily employed to examine the areas between the teeth, known as interproximal spaces. These spaces are difficult to visualize during a clinical examination but are prone to developing dental caries. By using BW dental x-rays 420, dentists can detect cavities, assess the extent of decay, evaluate the health of the supporting bone, and monitor the fit of dental restorations in the posterior teeth. An important aspect of the invention is that a measured distance on the BW dental x-ray 420 from the identified CEJ to the first intersection of alveolar bone and tooth or the identified apex of an infrabony pocket must both be located on the same side of the tooth. A side being one of: a mesial 310, a distal 320, a buccal 330, a lingual 340, a facial 350 surface of the tooth. An additional important aspect of the invention is that a measured distance on the PA dental x-ray 410 from the identified CEJ to the first intersection of alveolar bone and tooth or the identified apex of an infrabony pocket and measure a distance on the PA dental x-ray 410 from the identified CEJ to the identified apex of root must both be located on the same side of the tooth. A side being one of: a mesial 310, a distal 320, a buccal 330, a lingual 340, a facial 350 surface of the tooth. It should also be understood an algorithm may also format a PA dental image surface of a tooth which may include a patient data. Receive a patient data and associate with the PA dental image and the bone level percentage measurement to produce a correlated information. Wherein the algorithm may format the PA dental image based on another algorithm associated with tooth surface processing; merge the formatted PA dental image to the correlated dental information; identify and correct a discrepancy between the correlated information and the tooth surface processing; and associate the correlated information with a PA dental image. It should also be understood an algorithm may also format a BW dental image surface of a tooth which may include a patient data. Receive a patient data and associate with the BW dental image and at least one of: the bone level percentage measurement, the percentage to produce a correlated information. Wherein the algorithm may format the BW dental image based on another algorithm associated with tooth surface processing; merge the formatted BW dental image to the correlated dental information; identify and correct a discrepancy between the correlated information and the tooth surface processing; and associate the correlated information with a BW dental image. Wherein the tooth surface processing identifies a side of a tooth as being one of: a mesial 310, a distal 320, a buccal 330, a lingual 340, a facial 350 surface of the tooth. The algorithm may also be configured to identify more than one surface of the tooth.

The system or method may include at least one of: a server, a processor, a microprocessor, a processing device receives a dental image from a user or provider. Examples of a dental image user or provider may include: a patient, a dentist, a doctor, an insurance company, a bioinformatics service, a business, an e-commerce service, a ML service, among others. Further, the dental image may be obtained from at least one of: a dental x-ray, a digital x-ray, a digital image, a cell phone captured image, a photographic image, a toothbrush with an imaging device, a toothbrush with an imaging device being a camera, a film based x-ray, a digitally scanned x-ray, a digitally captured x-ray, a scintillator technology based image, a trans-illumination image, a fluorescence technology based image, a blue fluorescence technology based image, a laser based technology based image, a magnetic resonance image (MRI), a cone beam computed tomography (CBCT), a computed tomography (CT) scan based image of a section and/or an entirety of a mouth of a patient, and all future embodiments. A dental x-ray may also be referred as a dental image, a PA dental image, a BW dental image, panoramic x-ray, cephalometric dental x-ray.

At least one of: a server, a processor, a microprocessor, a processing device may use one or more ML algorithms to calculate bone level measurements on dental images. The dental image may be processed with a heuristic periapical bitewing algorithm to classify a dental image as one of: a PA dental image, a BW dental image, a non-dental image. Wherein a non-dental image is replaced with a notification. Bone level measurements for PA dental images may be calculated as follows. The PA dental image may be processed with a ML CEJ algorithm. The PA dental image may also be processed with a ML first intersection of coronal alveolar bone and tooth algorithm and a ML apex of root algorithm. At least one of: a server, a microprocessor, a processor, a processing device may identify a CEJ on the PA dental image with the ML CEJ algorithm. At least one of: a server, a microprocessor, a processor, a processing device may identify a first intersection of coronal alveolar bone and tooth on the PA dental image with the ML first intersection of coronal alveolar bone and tooth algorithm. At least one of: a server, a processor, a microprocessor, a processing device may identify an apex of root on the PA dental image with the ML apex of root algorithm. The algorithms are configured to measure the shortest contiguous distance on the PA dental image from the identified CEJ to the identified first intersection of coronal alveolar bone and tooth and then measure the shortest contiguous distance on the PA dental image from the identified CEJ to an identified apex of root. The algorithm further configured to at least one of: process and calculate, derive a quantitative score from the measured shortest contiguous distance on the PA dental image from the identified CEJ to the identified first intersection of coronal alveolar bone and tooth (numerator) and divide by the measured shortest contiguous distance on the PA dental image from the identified CEJ to the identified apex of root (denominator) to produce at least one of: a bone level percentage, a measurement. A bone level percentage may be expressed as a decimal, a quotient, a ratio, a whole number, or a percentage, a proportion, an average, a median, a standard deviation. The algorithm is configured to associate the bone level percentage measurement with the PA dental image. It should be understood that numerator and denominator are mathematical terms to describe fraction notation. As such the numerator is always the top number in a fraction and the denominator is always the bottom number in a fraction. This invention adheres to the accepted mathematical nomenclature of a fraction. Mathematical definitions of a numerator and a denominator in this document may be referenced in Bittinger, M., Ellenbogen, D., Johnson, B. (2017). Elementary and Intermediate Algebra: Concepts and Applications (7^{th} ed.). Pearson.

Bone level measurements for BW dental images may be calculated as follows. The BW dental image may be processed with a ML CEJ algorithm. The BW dental image may also be processed with a ML first intersection of coronal alveolar bone and tooth algorithm. At least one of: a server, a processor, a microprocessor, a processing device may identify a CEJ on the BW dental image with the ML CEJ algorithm. At least one of: a server, a processor, a microprocessor, a processing device may identify a first intersection of coronal alveolar bone and tooth on the BW dental image with the ML first intersection of coronal alveolar bone and tooth algorithm. The algorithms are configured to measure the shortest contiguous distance on the BW dental image from the identified CEJ to the identified first intersection of coronal alveolar bone and tooth. The algorithm further configured to process and calculate: the measured shortest contiguous distance on the BW dental image from the identified CEJ to the identified first intersection of coronal alveolar bone and tooth and process with an image aspect ratio algorithm to derive a bone level percentage or measurement. A bone level percentage or measurement may be expressed as a decimal, a quotient, a ratio, a percentage, a whole number, a proportion, an average, a median, a standard deviation. The algorithm is configured to associate the bone level percentage measurement with the BW dental image. Wherein an algorithm is also configured to compensate for a location variance of at least one of: the identified CEJ, the identified first intersection of coronal alveolar bone and tooth, the identified apex of root. The bone level percentage, quotient, ratio, a whole number, a proportion, an average, a median, a standard deviation, measurement and the PA or BW dental image may be displayed on a user interface such as a GUI. Wherein a measurement may be a quantitative score. Wherein the shortest contiguous distance may be expressed as at least one of: a curve, a straight line. It is possible for the shortest contiguous distance to be both a straight line and a curved line.

Bone level measurements on dental images with ML algorithms may also be measured on panoramic dental images. Bone level measurements for panoramic dental image may be calculated as follows. A least one of: a server, a processor, a microprocessor, a processing device may receive a panoramic dental image dental image from a user or provider. A heuristic periapical bitewing algorithm may be further configured to classify a dental image as one of: a periapical (PA) dental image, a bitewing (BW) dental image, panoramic image, a cephalometric image, a non-dental image. Wherein a non-dental image is a discrepancy. Wherein a non-dental image is replaced with a user or provider notification. A panoramic dental image may be processed with a ML CEJ algorithm. A panoramic dental image may be processed with a ML first intersection of coronal alveolar bone and tooth algorithm and a ML apex of root algorithm. At least one of: a server, a processor, a microprocessor, a processing device may identify a CEJ on a panoramic dental image with the ML CEJ algorithm. At least one of: a server, a processor, a microprocessor, a processing device may identify a first intersection of coronal alveolar bone and tooth on a panoramic dental image with the ML first intersection of coronal alveolar bone and tooth algorithm. At least one of: a server, a processor, a microprocessor, a processing device may identify an apex of root on a panoramic dental image with the ML apex of root algorithm. The algorithms are configured to measure the shortest contiguous distance on a panoramic dental image from the identified CEJ to the identified first intersection of coronal alveolar bone and tooth and then measure the shortest contiguous distance on a panoramic dental image from the identified CEJ to an identified apex of root. The algorithm further configured to process and calculate: the measured shortest contiguous distance on the PA dental image from the identified CEJ to the identified first intersection of coronal alveolar bone and tooth (numerator) and divide by the measured shortest contiguous distance on the PA dental image from the identified CEJ to the identified apex of root (denominator) to produce a bone level percentage measurement. A bone level percentage may be expressed as a decimal, a quotient, a ratio, a whole number, a percentage, a proportion, an average, a median, a standard deviation. The algorithm is configured to associate the bone level percentage measurement with a panoramic dental image.

Bone level measurements on dental images with ML algorithms may also be measured on cephalometric dental images. Bone level measurements for cephalometric dental image may be calculated as follows. A least one of: a server, a processor, a microprocessor, a processing device may receive a cephalometric dental image from a user or provider. A heuristic periapical bitewing algorithm may be further configured to classify a dental image as one of: a periapical (PA) dental image, a bitewing (BW) dental image, panoramic image, a cephalometric image, a non-dental image. Wherein a non-dental image is a discrepancy. Wherein a non-dental image is replaced with a user or provider notification. A cephalometric dental image may be processed with a ML CEJ algorithm. A cephalometric dental image may be processed with a ML first intersection of coronal alveolar bone and tooth algorithm and a ML apex of root algorithm. At least one of: a server, a processor, a microprocessor, a processing device may identify a CEJ on a cephalometric dental image with the ML CEJ algorithm. At least one of: a server, a processor, a microprocessor, a processing device may identify a first intersection of coronal alveolar bone and tooth on a cephalometric dental image with the ML first intersection of coronal alveolar bone and tooth algorithm. At least one of: a server, a processor, a microprocessor, a processing device may identify an apex of root on a cephalometric dental image with the ML apex of root algorithm. The algorithms are configured to measure the shortest contiguous distance on a cephalometric dental image from the identified CEJ to the identified first intersection of coronal alveolar bone and tooth and then measure the shortest contiguous distance on a cephalometric dental image from the identified CEJ to an identified apex of root. The algorithm further configured to process and calculate: the measured shortest contiguous distance on the PA dental image from the identified CEJ to the identified first intersection of coronal alveolar bone and tooth (numerator) and divide by the measured shortest contiguous distance on the PA dental image from the identified CEJ to the identified apex of root (denominator) to produce a bone level percentage measurement. A bone level percentage may be expressed as a decimal, a quotient, a ratio, a whole number, a percentage, a proportion, an average, a median, a standard deviation. The algorithm is configured to associate the bone level percentage measurement with a cephalometric dental image.

An important aspect of the invention is that at least one of: a server, a processor, a microprocessor, a processing device may execute an instruction in any order. Another aspect is that at least one of: a server, a processor, a microprocessor, a processing device is configured to compensate for at least one of: a distorted information, a missing image information on the dental image. Further at least one of: a server, a processor, a microprocessor, a processing device may omit the identified CEJ that is in the mandibular bone and/or omit the identified CEJ that is in the maxillary bone. The invention includes a user interface for displaying at least one of: the identified CEJ, the identified first intersection of coronal alveolar bone and tooth, an identified apex of infrabony pocket, an identified apex of root, the shortest contiguous distance on the PA dental image from the identified CEJ to the identified first intersection of coronal alveolar bone and tooth, the shortest contiguous distance on the PA dental image from the identified CEJ to the identified apex of root, the bone level percentage measurement of a PA dental image, a patient data associated with a PA dental image, the shortest contiguous distance on the BW dental image from the identified CEJ to the identified first intersection of coronal alveolar bone and tooth, the bone level percentage or measurement of a BW dental image, a patient data associated with a PA or BW dental image, the bone level measurement, the bone level percentage or measurement, the PA dental image, the BW dental image, the patient data, a correlated dental information, correlation dataset for a user or provider to view.

At least one of: a server, a processor, a microprocessor, a processing device may be configured to receive a dental image and identify if the dental image is a PA dental image or a BW dental image. This identification may be controlled by a heuristic periapical bitewing algorithm. The heuristic periapical bitewing algorithm may be one or more algorithms. A dental image process with a heuristic periapical bitewing algorithm will be classified as one of: a PA dental image, a BW dental image. If the heuristic periapical bitewing algorithm determines the image is a BW image at least one of: a server, a processor, a microprocessor, a processing device is configured to process the BW dental image as follows. The BW dental image may be processed with a ML CEJ algorithm. The BW dental image may also be processed with a ML first intersection of coronal alveolar bone and tooth algorithm. At least one of: a server, a processor, a processing device may identify a CEJ on the BW dental image with the ML CEJ algorithm. At least one of: a server, a processor, a processing device may identify a first intersection of coronal alveolar bone and tooth on the BW dental image with the ML first intersection of coronal alveolar bone and tooth algorithm. The algorithms are configured to measure the shortest contiguous distance on the BW dental image from the identified CEJ to the identified first intersection of coronal alveolar bone and tooth and process with an image aspect ratio algorithm to derive a bone level percentage or measurement. Associate the identified CEJ, the identified first intersection of coronal alveolar bone and tooth, bone level percentage or measurement with the BW dental image. The BW dental image may be associated with a patient data. The associated BW dental image may be provided to a user interphase or GUI.

At least one of: a server, a processor, a microprocessor, a processing device may query and receive a patient data. Wherein at least one of: a server, a processor, a microprocessor, a processing device may associate the dental image with a patient data. Further at least one of: a server, a processor, a microprocessor, a processing device may be configured to store a patient data with at least one of: an identified CEJ, an identified first intersection of coronal alveolar bone and tooth, an identified apex of root, an identified apex of infrabony pocket, the shortest contiguous distance on the PA dental image from the identified CEJ to the identified first intersection of coronal alveolar bone and tooth, the shortest contiguous distance on the PA dental image from the identified CEJ to an identified apex of root, the bone level percentage measurement of a PA dental image, the bone level measurement, the bone level percentage or measurement, the PA dental image, the BW dental image, the patient data, a correlated dental information, correlation dataset. At least one of: a server, a processor, a microprocessor, a processing device is further configured to compensate for a missing component of the patient data. A patient data may also be obtained from a bioinformatics organization that provides a dataset that may include a gene, a gene identifier, a gene sequence, a single nucleotide polymorphism, a nucleic acid sequence, a protein sequence (proteomics), an annotating genome, a shotgun sequence, a disease, a caries susceptibility, an impacted tooth, a tooth loss, an angle's classification of malocclusion, an immunoglobulin G (IGG) level, an immunoglobulin A (IGA) level, an immunoglobulin level, among others. Further this patient data associated with a bioinformatics dataset may be associated with at least one of: the identified CEJ, the identified first intersection of coronal alveolar bone and tooth, an identified apex of infrabony pocket, the identified apex of root, the shortest contiguous distance on the PA dental image from the identified CEJ to the identified first intersection of coronal alveolar bone and tooth, an apex of infrabony pocket, the shortest contiguous distance on the PA dental image from the identified CEJ to an identified apex of root, the bone level percentage measurement of a PA dental image, the bone level measurement, the bone level percentage or measurement, the PA dental image, the BW dental image, the patient data, a correlated dental information, correlation dataset.

Another embodiment of the present invention is a system or method for providing an anatomic delineation percentage measurement of a dental image, comprising at least one of: a server, a processor, a microprocessor, a processing device configured to receive a dental image from a provider or user and execute an instruction in any order. Wherein the dental image is obtained from an image capture device. At least one of: a server, a processing, a microprocessor, a processing device may process the dental image using at least one ML algorithm: comprising a ML anatomy algorithm, a ML pathology algorithm to identify anatomic delineations in the dental image. Measure the distance on the dental image between a first identified anatomic delineation and a second identified anatomic delineation and then the distance on the dental image between a first identified anatomic delineation and a third identified anatomic delineation. Calculate the anatomic delineation percentage measurement by dividing the distance on the dental image between the first and second identified anatomic delineations (numerator) by the distance on the dental image between the first and third identified anatomic delineations (denominator). An anatomic delineation percentage measurement may be expressed as at least one of: a decimal, a quotient, a ratio, a whole number, a percentage, a proportion, an average, a median, a standard deviation. This calculation may also be approached by calculating the distance on the dental image between the first identified anatomic delineation and the second identified anatomic delineation and the distance on the dental image between first identified anatomic delineation and the third identified anatomic delineation and deriving a percentage. Associate the derived percentage as at least one of: a decimal, a quotient, a ratio, a whole number, a percentage, a proportion, an average, a median, a standard deviation measurement with the dental image. The processing device may be configured with a user interface for displaying the derived percentage and/or the anatomic delineation percentage measurement(s) with a dental image which may include a patient data for a user or provider. It should be understood anatomic delineations can have specific anatomic locations, wherein the first identified anatomic delineation is an identified CEJ, wherein a second identified anatomic delineation is an identified first intersection of coronal alveolar bone and tooth, wherein a third identified anatomic delineation is an identify apex of root.

The invention will also be configured to differentiate between a PA dental image and a BW dental image. Wherein, dental image may be one or more of: an x-ray, a digital image, and future dental imaging techniques. This differentiation will be controlled by a heuristic periapical bitewing algorithm. The heuristic periapical bitewing algorithm may be one or more algorithms.

An additional embodiment of the present invention is further processing a dental image with a heuristic periapical bitewing algorithm. If the algorithm determines the image is a BW dental image at least one of: a server, a processor, a processing device is configured to provide an anatomic delineation percentage measurement of the BW dental image. The system or method may comprise at least one of: a server, a processor, a microprocessor, a processing device configured to receive a dental image from a provider or user and execute an instruction in any order. Wherein the dental image is obtained from an image capture device or memory. The dental image may be processed with the heuristic periapical bitewing algorithm to classify a dental image as one of: a PA dental image, a BW dental image. A bone level measurement of a BW dental image may be calculated as follows. At least one of: a server, a processor, a microprocessor, a processing device may process the BW dental image using at least one ML algorithm: comprising a ML anatomy algorithm, a ML pathology algorithm to identify anatomic delineations in the BW dental image. Measure the distance on the dental image between a first identified anatomic delineation and a second identified anatomic delineation. Calculate the anatomic delineation percentage measurement by measuring the distance on the dental image between the first and second identified anatomic delineations and process with an image aspect ratio algorithm to produce at least one of: a percentage, a measurement. An anatomic delineation percentage measurement may be expressed as a decimal, a quotient a ratio, a whole number, a percentage, a proportion, an average, a median, a standard deviation. This calculation may also be approached by calculating the distance on the dental image between the first identified anatomic delineation and the second identified anatomic delineation and processing with an image aspect ratio algorithm and deriving a percentage. Associate the derived percentage as at least one of: a decimal, a quotient, a ratio, a whole number, a percentage, a proportion, an average, a median, a standard deviation measurement with the BW dental image. The processing device may be configured with a user interface for displaying the derived percentage and/or the anatomic delineation percentage measurement(s) with the BW dental image which may include a patient data. It should be understood anatomic delineations can have specific anatomic locations, wherein the first identified anatomic delineation is an identified CEJ, wherein the second identified anatomic delineation is an identified first intersection of coronal alveolar bone and tooth.

An addition to this embodiment is that the processing device may calculate the anatomic delineation percentage measurements using a weighted average of distances on the dental image between the identified anatomic delineations. Further, at least one of: a server, a processor, a microprocessor, a processing device is configured to apply image preprocessing techniques to the dental image before processing with ML algorithms. The processing device is also configured to compensate for at least one of: a distorted information, a missing image information on the dental image. It should be understood that the processing device may identify the first, second, and third anatomic delineations using a combination of the ML anatomy and pathology algorithms. It should also be understood an algorithm may also format a dental image surface of a tooth which may include a patient data. Receive a patient data and associate with the dental image and the anatomic delineation percentage measurement to produce a correlated information. Wherein an anatomic delineation measurement may be a quantitative score. Wherein the algorithm may format the dental image based on another algorithm associated with tooth surface processing; merge the formatted dental image to the correlated dental information; identify and correct a discrepancy between the correlated information and the tooth surface processing; and associate the correlated information with the dental image.

Wherein the tooth surface processing identifies a side of a tooth as being one of: a mesial 310, a distal 320, a buccal 330, a lingual 340, a facial 350 surface of the tooth. The algorithm may also be configured to identify more than one surface of the tooth.

At least one of: a server, a processor, a microprocessor, a processing device may associate the dental image with a patient data. Wherein a patient data may include personal information such as an age, a first name, a gender, a middle initial, a last name, a date of birth, a zip code, an address, a cell phone number, a land line number, a current medication, a previous medication, a social security number, a marital status, an insurance, an e-commerce consumer's insurance identification number, a change of insurance, a change of employment, a change of zip code, a change of the previous medication, a change of the marital status, a change of the gender, among others. At least one of: a server, a processor, a processing device may associate a patient data and a dental image with a regulatory policy. It may further verify a patient data and/or a dental image is compliant with a regulator policy with a patient notification. A regulatory policy may include the health insurance portability and accountability act (HIPAA), an end user licensing agreement (EULA), a licensing agreement (SLA), a security token, a swipe authorization, a signed consent form, among others. At least one of: a server, a processor, a processing device may identify at least one of: a patient data, a dental image that is noncompliant with a regulator policy and send a notification to at least one of: a user, a provider, an e-commerce organization, a ML entity, an algorithm.

A patient data may also be obtained from a bioinformatics company, bioinformatics organization that provides a bioinformatics dataset. A bioinformatics dataset may include a gene, a gene identifier, a gene sequence, a single nucleotide polymorphism, a nucleic acid sequence, a protein sequence (proteomics), an annotating genome, a shotgun sequence, a disease, a caries susceptibility, an impacted tooth, a tooth loss, an angle's classification of malocclusion, an immunoglobulin G (IGG) level, an immunoglobulin A (IGA) level, an immunoglobulin level, among others.

Another source of a patient data may be obtained from an insurance company or an insurance organization that may provide an insurance dataset. An insurance dataset may include an American Dental Association (ADA) code, a date, an insurance claim identifier, an insurance claim number, an insurance claim, multiple or duplicate claims, a national provider identification number for a provider or an institution and/or a provider's state license number, a data among others.

In yet another embodiment of the present invention is a system or method for providing a bone level percentage measurement of a dental image is discussed. The system or method may comprise one or more of: a server, a processor, a microprocessor, a processing device which are configured to receive a dental image of a patient from a dental image user or provider and process an instruction in any order. At least one of: a server, a processor, a microprocessor, a processing device may be configured to differentiate between a PA dental image and a BW dental image with a heuristic periapical bitewing algorithm. The dental image may be processed with the heuristic periapical bitewing algorithm to classify a dental image as one of: a PA dental image, a BW dental image. PA dental images will be used to calculate bone level measurements as follows. Process the PA dental image with at least one algorithm comprising: a ML CEJ algorithm, a ML first intersection of coronal alveolar bone and tooth algorithm, a ML apex of root algorithm. Identify a CEJ on the PA dental image with the ML CEJ algorithm to produce an identified CEJ. Identify the first intersection of coronal alveolar bone and tooth on the PA dental image with the ML first intersection of coronal alveolar bone and tooth algorithm to produce an identified first intersection of coronal alveolar bone and tooth. Identify an apex of root on the PA dental image with the ML apex of root algorithm to produce an identified apex of root. Measure a distance on the PA dental image from the identified CEJ to the identified first intersection of coronal alveolar bone and tooth. Measure a distance on the PA dental image from the identified CEJ to the identified apex of root. From the PA dental image, process and calculate the distance from the identified CEJ to the identified first intersection of coronal alveolar bone and tooth and divide by the distance from the identified CEJ to the identified apex of root to produce the bone level percentage measurement. Identify a PA dental image missing at least one of: a CEJ 150, a first intersection of coronal alveolar bone and tooth 160, an apex of root 190 and instruct at least one of: a server, a processor, a microprocessor, a processing device to at least one of: omit, process, reclassify the calculation. Another approach is to calculate the distance between the identified CEJ and the identified first intersection of coronal alveolar bone and tooth and between the identified CEJ and the apex of root to derive the percentage of bone loss by calculating a ratio of the distance. An important aspect of the invention is to identify a PA dental image with a distorted or missing view of at least one of: a CEJ 150, a first intersection of coronal alveolar bone and tooth 160, an apex of root 190 and instruct at least one of: a server, a processor, a microprocessor, a processing device to at least one of: omit, process, reclassify the calculation associated with the PA dental image. Query and receive a patient data of the patient associated with the dental image. Associate at least one of: the identified CEJ, the identified first intersection of coronal alveolar bone and tooth, an identified apex of infrabony pocket, the identified apex of root, the bone level percentage measurement with the patient data to produce a correlated dental information or a correlated dataset. Provide the correlated dental information or correlated dataset to a memory.

The invention which may use at least one of: a server, a processor, a microprocessor, a processing device is configured to identify a PA dental image missing at least one of: the CEJ 150, the first intersection of coronal alveolar bone and tooth 160, the apex of root 190 and instruct the processor to at least one of: omit, process, reclassify the calculation. The invention may also be configured to identify a PA dental image with an obstructed view of at least one of: the identified CEJ, the identified first intersection of coronal alveolar bone and tooth, an identified apex of infrabony pocket, the identified apex of root and instruct the processor to at least one of: omit, process, reclassify the calculation. Further, the invention is configured to compensate for least one of: the CEJ 150, the first intersection of alveolar bone 160, an identified apex of infrabony pocket, the apex of root 190 that is missing or obstructed. A server, a processor, microprocessor, a processing device includes an image processing algorithm(s) or an image processing component configured to: compensate for at least one of: a distorted image information, a missing image information, an obstructed image information.

Another aspect of the invention includes at least one of: a server, a processor, a microprocessor, a processing device which are configured to use a GUI for displaying at least one of: said PA dental image, said identified CEJ, said first intersection of coronal alveolar bone and tooth, said identified apex of infrabony pocket, said identified apex of root, said measuring the shortest contiguous distance on the PA dental image from said identified CEJ to said identified apex of root, said shortest contiguous distance on said BW dental image from said identified CEJ to said identified first intersection of coronal alveolar bone and tooth, said bone level percentage measurement, said measurement, said patient data, said correlated dental information, said correlation dataset.

Additional aspects of the invention will now be discussed. The dental image user or provider may obtain a dental image from at least one of: an image capture device, a data storage device. At least one of: a server, a processor, a microprocessor, a processing device is configured to compensate for a missing component of said patient data. Wherein said server, said processor, said microprocessor, said processing device may be configured to identify said identified CEJ at any location within said CEJ and may be configured to identify said identified first intersection of coronal alveolar bone and tooth at any location on said alveolar bone and may be configured to identify said identified apex of root at any location on the tooth apex a root. At least one of: a server, a processor, a microprocessor, a processing device may omit the identified CEJ that is in the mandibular bone and may omit the identified CEJ that is in the maxillary bone. Wherein at least one of: a server, a processor, a microprocessor, a processing device is configured to calculate on said PA dental image said bone level percentage measurement with said identified CEJ, said identified first intersection of coronal alveolar bone and tooth and said identified apex of root that are all located on one of: a mesial 310, a distal 320, a buccal 330, a lingual 340, a facial 350 surface of the tooth. Another perspective is at least one of: a server, a processor, a microprocessor, a processing device is configured to calculate on said PA dental image said bone level percentage measurement from said identified CEJ and said identified first intersection of coronal alveolar bone and tooth, and said identified apex of root which are all located on the same surface of the tooth. Further, at least one of: a server, a processor, a microprocessor, a processing device is configured to calculate on said BW dental image at least one of: said bone level percentage measurement, said measurement with said identified CEJ, said identified first intersection of coronal alveolar bone and tooth and said identified apex of root that are all located on one of: a mesial 310, a distal 320, a buccal 330, a lingual 340, a facial 350 surface of the tooth. Another perspective is at least one of: a server, a processor, a microprocessor, a processing device is configured to calculate on said BW dental image at least one of: said bone level percentage measurement, said measurement from said identified CEJ and said identified first intersection of coronal alveolar bone and tooth which are all located on the same surface of the tooth. Wherein the same surface of the tooth is one of: a mesial 310, a distal 320, a buccal 330, a lingual 340, a facial 350 surface of the tooth. Wherein at least one of: the shortest contiguous distance on the PA dental image from the identified CEJ to the identified first intersection of coronal alveolar bone and tooth, the shortest contiguous distance on the PA dental image from the identified CEJ to the identified apex of root are only located on at one of: a mesial 310, a distal 320, a buccal 330, a lingual 340, a facial 350 surface of the tooth. Wherein the shortest contiguous distance on the BW dental image from the identified CEJ to the identified first intersection of coronal alveolar bone and tooth is only located on at one of: a mesial 310, a distal 320, a buccal 330, a lingual 340, a facial 350 surface of the tooth. More specifically, wherein the processor is configured to calculate the bone level percentage measurement on a PA dental image from the identified CEJ, and the identified first intersection of coronal alveolar bone and tooth, and the identified apex of root that are all located on the same surface of the tooth; wherein the microprocessor is configured to calculate at least one of: a bone level percentage measurement, a measurement on a BW dental image from the identified CEJ, and the identified first intersection of coronal alveolar bone and tooth that are all located on the same surface of the tooth; wherein the same surface of the tooth is one of: a mesial, a distal, a buccal, a lingual, a facial surface of the tooth.

In another aspect of the invention an algorithm may also format a PA dental image based on another algorithm associated with tooth surface processing as follows: format the PA dental image based on another algorithm associated with tooth surface processing; on the formatted PA dental image calculate a formatted bone level percentage measurement from the identified CEJ, and the identified first intersection of coronal alveolar bone and tooth, and the identified apex of root that are all located on the same surface of the tooth; identify and notify a discrepancy between the formatted bone level percentage measurement and at least one of: the CEJ, the first intersection of coronal alveolar bone and tooth, the apex of root. Associate the formatted bone level percentage measurement with the PA dental image. It should also be understood an algorithm may also format a BW dental image based on another algorithm associated with tooth surface processing as follows: format the BW dental image based on another algorithm associated with tooth surface processing; on the formatted BW dental image calculate at least one of: a formatted bone level percentage measurement, a formatted measurement from the identified CEJ, and the identified first intersection of coronal alveolar bone and tooth that are all located on the same surface of the tooth; identify and notify a discrepancy between at least one of: the formatted bone level measurement, the measurement and at least one of: the CEJ, the first intersection of coronal alveolar bone and tooth. Associate at least one of: the formatted bone level measurement, the formatted measurement with the BW dental image. Further at least one of: a server, a processor, a microprocessor, a processing device may also identify whether the patient is informed in regards to a notification obligation such as a HIPAA, an EULA, a SLA, a security token, a swipe authorization, a signed consent form, a regulatory notification among others to process and/or analyze the dental image and the patient data.

A dental image may be acquired from an image capture device and/or a storage device. An image capture device may include: an x-ray equipment, a digital camera, a cell phone camera, an indirect or direct flat panel detector (FPD), a charged couple device (CCD), a phosphor plate radiography device, a picture archiving and communication system (PACS), a photo-stimulable phosphor (PSP) device, a computer tomography (CT) device, a wireless complementary metal-oxide-semiconductor (CMOS), a cone beam computed tomography (CBCT) device, and all future embodiments. The dental image may be provided to the invention by a user or a provider. An example of a user or a provider may include a patient, a dentist, a doctor, an insurance company, an insurance organization, a bioinformatics company, a business, a bioinformatics organization, an e-commerce service, a ML company a cloud based company, among others. A storage device may include volatile and nonvolatile, removable, and non-removable media implemented in any method or technology for storage of information, such as computer-readable instructions, data structures, program modules, all future embodiments of storage devices.

A bone level percentage may be expressed as at least one of: a decimal, a quotient, a percentage, a ratio, a whole number, a proportion, an average, a median, a standard deviation and may be displayed on a user interface such as a GUI. An example of a GUI include a desktop computer, a workstation, a laptop computer, a cell phone, a tablet, a mobile device, a monitor, display screen, among others. A GUI may also interact with the client application(s) on the client device with a keyboard based input, a mouse based input, a voice based input, a pen based input, and a gesture based input, among others. The gesture based input may include one or more touch based actions such as a touch action, a swipe action, and a combination of each, among others. Processing a dental image with an undescribed hardware may disrupt the invention's ability to process bone level measurements on dental images with ML algorithms. Wherein at least one of: a server, a processor, a microprocessor, a processing device will be programed to omit processing on an undescribed hardware and request processing with a described hardware and continue processing. Wherein a described hardware includes at least one of: a server, a processor, microprocessor, a processing device, client device, a keyboard based input, a mouse based input, a voice based input, a pen based input, and a gesture based input, a monitor, a cell phone, a computer, a tablet computer, a laptop computer, user interphase, a GUI.

Furthermore a cluster analysis may be performed on at least one of: the identified CEJ, the identified first intersection of coronal alveolar bone and tooth, an identified apex of infrabony pocket, an identified apex of root, the shortest contiguous distance on the PA dental image from the identified CEJ to the identified first intersection of coronal alveolar bone and tooth, the shortest contiguous distance on the PA dental image from the identified CEJ to the identified apex of root, the bone level measurement, the bone level percentage or measurement, the PA dental image, the BW dental image, the patient data, a correlated dental information, correlation dataset, the bone level percentage measurement of a PA dental image, the bone level percentage or measurement, BW dental image based on at least one or more of: a spatial detection, a sequential pattern mining, dataset(s) comparison, a data analysis, a statistical data analysis, a Boolean Logic analysis, a fuzzy logic analysis, a machine learned analysis, an anomaly detection analysis mechanism, among others.

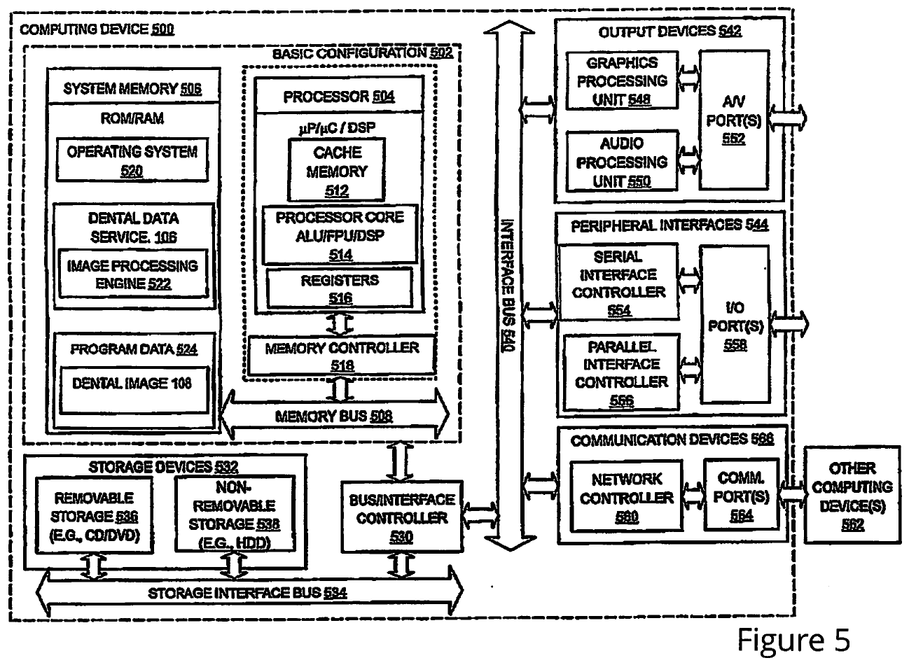

FIG. 5 is an example of a computing device 500. Wherein a computing device is at least one of: a server, a processor, a microprocessor, a processing device. Depending on the desired configuration, the processor 504 may be of any type, including but not limited to a server, a processor, a microprocessor (μP), a processing device, a microcontroller (μC), a digital signal processor (DSP), or any combination thereof. The processor 504 may include one more levels of caching, such as a level cache memory 512, one or more processor cores 514, and registers 516. The example processor cores 514 may (each) include an arithmetic logic unit (ALU), a floating-point unit (FPU), a digital signal processing core (DSP Core), a graphics processing unit (GPU), or any combination thereof. An example memory controller 518 may also be used with the processor 504, or in some implementations, the memory controller 518 may be an internal part of the processor 504.

Depending on the desired configuration, the system memory 506 may be of any type including but not limited to volatile memory (such as RAM), non-volatile memory (such as ROM, flash memory, etc.), or any combination thereof. The system memory 506 may store and provide an operating system 520, with at least one of: a server, a processor, a microprocessor, a processing device and a program data 524. At least one of: a server, a processor, a microprocessor, a processing device may include components such as an image processing engine 522. The image processing engine 522 may execute the instructions and processes associated with at least one of: a processor, a microprocessor, a processing device, a server. In an example scenario, the image processing engine 522 may receive a dental image of a patient from a user or provider.

Input to and output out of at least one of: a server, a processor, a microprocessor, a processing device may be transmitted through a communication device 566 that may be communicatively coupled to the computing device 500. A computing device 500 may include at least one of: a server, a processor, a microprocessor, a processing device. The communication device 566 may provide wired and/or wireless communication. The program data 524 may also include, among other data, the dental image for a user or provider, or the like, as described herein. The dental image may include an image and/or a digital image of dental structure(s) of a patient.

The computing device 500 may have additional features or functionality, and additional interfaces to facilitate communications between the basic configuration 502 and any desired devices and interfaces. For example, a bus/interface controller 530 may be used to facilitate communications between the basic configuration 502 and one or more data storage devices 532 via a storage interface bus 534. The data storage devices 532 may be one or more removable storage devices 536, one or more non-removable storage devices 538, a cloud storage device, or a combination thereof. Examples of the removable storage and the non-removable storage devices may include magnetic disk devices, such as flexible disk drives and hard-disk drives (FIDDs), optical disk drives such as compact disk (CD) drives or digital versatile disk (DVD) drives, solid state drives (SSDs), tape drives, flash memory, cloud based storage, a cloud computing platform providing a storage service, an open or a closed source platform providing a storage service, a virtual private network (VPN) providing a storage service, an ISO image disk, a cloud based storage service, a redundant array of independent disks (RAID), a USB based disk drive, a USB flash drive, a storage virtualization based storage service, a digital video service, a virtualized server providing a storage service, a super computer providing a storage service, a super computer parallel array providing a storage service, a dental practice management software providing a storage service, a dental digital image software providing a storage service, and/or all future embodiments. Example computer storage media may include volatile and nonvolatile, removable, and non-removable media implemented, cloud based storage in any method or technology for storage of information, such as computer-readable instructions, data structures, program modules, all future embodiments, or other data.

The system memory 506, the removable storage devices 536 and the non-removable storage devices 538 are examples of computer storage media. Computer storage media includes, but is not limited to, RAM, ROM, EEPROM, flash memory or other memory technology, cloud based memory, cloud based storage, CD-ROM, digital versatile disks (DVDs), solid state drives, or other optical storage, quantum memory, magnetic cassettes, magnetic tape, magnetic disk storage or other magnetic storage devices, or any other medium which may be used to store the desired information and which may be accessed by the computing device 500. Any such computer storage media may be part of the computing device 500.

The computing device 500 may also include an interface bus 540 for facilitating communication from various interface devices (for example, one or more output devices 542, one or more peripheral interfaces 544, and one or more communication devices 566) to the basic configuration 502 via the bus/interface controller 530. Some of the example output devices 542 include a graphics processing unit 548 or GUI, and an audio processing unit 550, which may be configured to communicate to various external devices such as a display, GUI, or speakers via one or more A/V ports 552. One or more example peripheral interfaces 544 may include a serial interface controller 554 or a parallel interface controller 556, which may be configured to communicate with external devices such as input devices (for example, keyboard, mouse, pen, voice input device, touch input device, etc.) or other peripheral devices (for example, printer, scanner, etc.) via one or more I/O ports 558. An example of the communication device(s) 566 includes a network controller 560, which may be arranged to facilitate communications with one or more other computing devices 562 over a network communication link via one or more communication ports 564. The one or more other computing devices 562 may include at least one of: a server, a processor, a microprocessor, a processing device, a computing device, and comparable devices.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) WiFi, Bluetooth, short range wireless interconnection, long range wireless interconnection, wireless networking technology, radio waves, light waves, any electromagnetic wave, and other wireless media. The term computer readable media as used herein may include both storage media and communication media.

The computing device 500 may be implemented as a part of a specialized server, mainframe, or similar computer, which includes any of the above functions. The computing device 500 may also be implemented on personal computer device(s) such as a laptop computer, a cell phone, and non-laptop computer configurations. Additionally, the computing device 500 may include specialized hardware such as an application-specific integrated circuit (ASIC), a field programmable gate array (FPGA), a programmable logic device (PLD), and/or a free form logic on an integrated circuit (IC), among others.

Additional example embodiments for a system or method to provide bone level measurements on dental images with ML algorithms are as follows. The system or method may include can be implemented in any number of ways, including or having structures described herein. One such way may be by machine operations, of devices of the type described in the present disclosure. Another optional way may be for one or more of the individual operations of the system or the method to be performed in conjunction with one or more human operators performing some of the operations while other operations may be performed by machines. These human operators need not be collocated with each other, but each can be only with a machine that performs a portion of the program. In other embodiments, the human interaction can be automated such as by pre-selected criteria that may be machine automated.

Furthermore, a dental image may be processed with a convolutional neural network (CNN). Wherein a convolutional neural network is an artificial neural network. Examples of CNN or an artificial neural network may include Regional based Convolutional Neural Networks (R-CNN), Fast Regional based Convolutional Neural Networks (Fast R-CNN), Faster Regional based Convolutional Neural Networks (Faster R-CNN) and masked R-CNN. R-CNN may use object bounding boxes, non-object bounding boxes, proposed regions, blobby images regions, a selective search, a support vector machine (SVM), a bounding box component, an object confidence score, an object classification, supervised training and unsupervised training to process dental images. Fast R-CNN may use a ROI pooling, bounding boxes, region proposals, a softmax layer, a bounding box component, an object confidence score, an object classification, supervised training and unsupervised training to process dental images. Faster R-CNN may use region proposal networks (RPN), bounding boxes, a softmax layer, anchors, a ROI pooling, ground truth boxes, a bounding box component, an object confidence score, an object classification, supervised learning and/or unsupervised learning to process dental images. Masked R-CNN may also be used for object detection, instance segmentation, and image segmentation tasks. Mask R-CNN is also used to identify and classify objects within an image, while also generating a pixel-level mask for each object that delineates its boundaries precisely.

An algorithm may merge and/or compare dental images to an object class dataset to generate an object confidence score and/or an object classification to produce a dental image probability map. At least one of: an object confidence score, an object classification, a dental landmark probability map of a dental image processed with CNN may be correlated and/or further merged with a correlation dataset or a correlated dental information. Wherein an algorithm may format the dental image based on a transaction processed by at least one of: a user, a provider, an e-commerce organization, a ML entity, an algorithm; merge the dental image with a transaction processed by at least one of: a user, a provider, an e-commerce organization, a ML entity, an algorithm into a correlated dental information and identify and correct a discrepancy between the dental image with a transaction processed by at least one of: a user, a provider, an e-commerce organization, a ML entity, an algorithm and the correlated dental information.

An instance segmentation process may be performed on a dental image with overlapping objects, multiple overlapping objects, different backgrounds, a Region of Interest Align (ROI Align), a class awareness, an instance awareness, an anchor box, a ground truth box, object confidence scores and binary masks generated for individual and/or multiple objects. Instance segmentation may be processed by Region proposed networks (RPN), Featured Pyramid Networks (FPN) and Fully Convolutional Networks (FCN). Dental image objects may also be processed in color, gray scale and black and white resolutions.

At least one of: an identified CEJ, an identified first intersection of coronal alveolar bone and tooth, an identified apex of infrabony pocket, an identified apex of root, the shortest contiguous distance on the PA dental image from the identified CEJ to the identified first intersection of coronal alveolar bone and tooth, the shortest contiguous distance on the PA dental image from the identified CEJ to the identified apex of root, a bone level percentage measurement of a PA dental image, the shortest contiguous distance on the BW dental image from the identified CEJ to the identified first intersection of coronal alveolar bone and tooth may be outlined or associated with one or more of: a pixel, a delineation, a straight line, a curved line, a circle, a square, a rectangle, a triangle, polygon, a key point, a polyline, a cuboid, a centroid on a dental image. The dental image may be processed with anchor boxes, ground truth boxes, landmarks, predictive landmarks, and a landmark probability map. One or more convolutional layers may be configured to extract predictive landmarks from the training of dental images. A predictive landmark probability may include an identified CEJ, an identified first intersection of coronal alveolar bone and tooth, an identified apex of infrabony pocket, an identified apex of root, the shortest contiguous distance on the PA dental image from the identified CEJ to the identified first intersection of coronal alveolar bone and tooth, the shortest contiguous distance on the PA dental image from the identified CEJ to the identified apex of root, the shortest contiguous distance on the BW dental image from the identified CEJ to the identified first intersection of coronal alveolar bone and tooth, a bone level percentage measurement, a bone level percentage or measurement, a first identified anatomic delineation, a second identified anatomic delineation, a third identified anatomic delineation, the distance between a first identified anatomic delineation and a second identified anatomic delineation, the distance between a first identified anatomic delineation and a third identified anatomic delineation, the anatomic delineation percentage measurement. A CNN or an artificial neural network may learn dental image class probabilities and dental object class probabilities from a dental image. Further a predictive landmark probability may be used to generate a landmark probability map from at least one of: an identified CEJ, an identified first intersection of coronal alveolar bone and tooth, an identified apex of infrabony pocket, an identified apex of root, the shortest contiguous distance on the PA dental image from the identified CEJ to the identified first intersection of coronal alveolar bone and tooth, the shortest contiguous distance on the PA dental image from the identified CEJ to the identified apex of root, the shortest contiguous distance on the BW dental image from the identified CEJ to the identified first intersection of coronal alveolar bone and tooth, a bone level percentage measurement, a bone level percentage or measurement, a first identified anatomic delineation, a second identified anatomic delineation, a third identified anatomic delineation, the distance between a first identified anatomic delineation and a second identified anatomic delineation, the distance between a first identified anatomic delineation and a third identified anatomic delineation, the anatomic delineation percentage measurement.

The CNN or artificial neural network may process the dental image with Euclidean geometry and/or extremely randomized forest functions to produce a dental image landmark probability or spatial relationships probability. A different dental image landmark probability is learned for each dental image landmark and the at least one of: a server, a processor, a microprocessor, a processing device may compensate for distorted and/or missing information. The convolutional neural network may then process the dental images with an image class and/or an object class with a ML spatial relationship function to determine the spatial relationship probabilities between the locations of the dental landmarks and produce a plurality of dental image landmark probability maps. Further the CNN or artificial neural network may machine learn object probability relationships between the locations of the dental images probability maps and machine learn object probability relationships between the locations of the dental image probability maps. At least one of: a server, a processor, a microprocessor, a processing device may provide a dental image landmark probability map and/or a spatial relationship probability demonstration aid from the plurality of dental image landmark probability to a user or provider. The demonstration aid may be displayed on a GUI. The processed landmark probability maps may be correlated to a correlation dataset or a correlated dental information and provided to a user, a provider, an e-commerce organization, a ML entity, an algorithm.

Metadata or other data associated with a received dental image may disrupt the invention's ability to process bone level measurements on dental images with ML algorithms. The invention will be programed to omit this data associated with a received dental image. The invention may replace this data or process this data with another data or algorithm and continue processing. Wherein at least one of: a server, a processor, a microprocessor, a processing device is configured to omit processing of a received dental image associated with at least one of: a metadata, a data, an algorithm, a dental image set or image set with a label, object sub-types, confidence scores, a probability value of an image class, an image class vector, an image class space, a field of view label, shallow hash neural network, hash neural network, laboratory records of a patient, laboratory test data, unified formats of lab test data, lab data off different formats a computer code, a computer data and replace the omit processing with at least one of: a data, an algorithm and continue processing. Wherein a lab is a laboratory. Wherein a label identifies a region of a particular anatomic structure. Further, at least one of: a server, a processor, a microprocessor, a processing device is configured to omit processing on an augmented reality display and request processing with a GUI. At least one of: a server, a processor, a microprocessor, a processing device is configured to omit processing of a provided dental image associated with at least one of: a dental image landmark probabilities dataset, an image class landmark probabilities dataset, an object class landmark probabilities dataset, a spatial landmark probability relationships dataset, an object probability landmarks dataset, an object probability relationships dataset, a dental image landmark probability map, a dental image landmark probability map dataset, a dental image dataset, a dataset and replace the omit processing with at least one of: a data, an algorithm and continue processing.

At least one of: a server, a processor, a microprocessor, a processing device may use a communication network to execute at least one of: a transfer, an exchange, a buy, a sell with at least one of: an identified CEJ, an identified first intersection of coronal alveolar bone and tooth, an identified apex of infrabony pocket, an identified apex of root, the shortest contiguous distance on the PA dental image from the identified CEJ to the identified first intersection of coronal alveolar bone and tooth, the shortest contiguous distance on the PA dental image from the identified CEJ to the identified apex of root, the bone level percentage measurement of a PA dental image, first identified anatomic delineation, a second identified anatomic delineation, a third identified anatomic delineation, an anatomic delineation percentage measurement, the bone level measurement, the bone level percentage or measurement, the PA dental image, the BW dental image, the patient data, a correlated dental information, correlation dataset; wherein a communication network includes at least one of: an internet, an intranet, an extranet, an internet transaction service, an online transaction service, a mobile network, a wireless network, an online transaction processing (OLTP) service, an online analytical processing (OLAP) service, a transaction platform, an internet transaction platform. Further, the communication network may exchange, transfer, buy, sell an identified CEJ, the identified first intersection of coronal alveolar bone and tooth, an identified apex of infrabony pocket, an identified apex of root, the shortest contiguous distance on the PA dental image from the identified CEJ to the identified first intersection of coronal alveolar bone and tooth, the shortest contiguous distance on the PA dental image from the identified CEJ to the identified apex of root, the bone level percentage measurement of a PA dental image, the PA dental image, the BW dental image, first identified anatomic delineation, a second identified anatomic delineation, a third identified anatomic delineation, an anatomic delineation percentage measurement, the bone level measurement, the bone level percentage or measurement, the PA dental image, the BW dental image, the patient data, a correlated dental information, correlation dataset, over at least one of: the internet, an intranet, an extranet, an internet, an internet transaction service, an online transaction service, a mobile network, a wireless network, an online transaction processing (OLTP) service, an online analytical processing (OLAP), a transaction platform. An additional aspect may use at least one of: a user, a provider, an e-commerce organization, a ML entity, an algorithm may transfer, exchange, buy, sell at least one of: a dental image, a correlated dental information, a correlation dataset over a communication network, wherein a communication network includes at least one of: an internet, an intranet, an extranet, an internet transaction service, an online transaction service, a mobile network, a wireless network, an online transaction processing (OLTP) service, an online analytical processing (OLAP) service, a transaction platform.

In an example scenario of an e-commerce transaction at least one of: a user, a provider, an e-commerce organization, a ML entity, an algorithm may at least one of: exchange, transfer, buy, sell at least one of: an identified CEJ, the identified first intersection of coronal alveolar bone and tooth, an identified apex of infrabony pocket, an identified apex of root, the shortest contiguous distance on the PA dental image from the identified CEJ to the identified first intersection of coronal alveolar bone and tooth, the shortest contiguous distance on the PA dental image from the identified CEJ to the identified apex of root, the bone level percentage measurement of a PA dental image, a first identified anatomic delineation, a second identified anatomic delineation, a third identified anatomic delineation, the distance between a first identified anatomic delineation and a second identified anatomic delineation, the distance between a first identified anatomic delineation and a third identified anatomic delineation, the anatomic delineation percentage measurement, the bone level measurement, the bone level percentage or measurement, the PA dental image, the BW dental image, the patient data, a correlated dental information, correlation dataset over a communication network. An e-commerce transaction may include one or more of: business to business (B2B), business to consumer (B2C), consumer to business (C2B), consumer to consumer (C2C), business to administration (B2A), consumer to administration (C2A), among others. The e-commerce processing function may process a transaction of at least one of: an identified CEJ, the identified first intersection of coronal alveolar bone and tooth, an identified apex of infrabony pocket, an identified apex of root, the shortest contiguous distance on the PA dental image from the identified CEJ to the identified first intersection of coronal alveolar bone and tooth, the shortest contiguous distance on the PA dental image from the identified CEJ to the identified apex of root, the bone level percentage measurement of a PA dental image, a first identified anatomic delineation, a second identified anatomic delineation, a third identified anatomic delineation, an anatomic delineation percentage measurement, the bone level measurement, the bone level percentage or measurement, the PA dental image, the BW dental image, the patient data, a correlated dental information, correlation dataset with at least one of: a user, a provider, an e-commerce organization, a ML entity, an algorithm. Wherein a transaction includes at least one of: business to business (B2B), business to consumer (B2C), consumer to business (C2B), consumer to consumer (C2C), business to administration (B2A), consumer to administration (C2A) transactions, among others.

The embodiments of the invention, example scenarios and schemas in FIGS. 1 through 5 are shown with specific components, data types, and configurations. Embodiments are not limited to systems according to these example configurations. The invention, bone level measurements on dental images with ML algorithms may be implemented in configurations employing fewer or additional components in applications and user interfaces. Furthermore, the example schema and components shown in FIGS. 1 through 5 and their subcomponents may be implemented in a similar manner with other values using the principles described herein.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

1. A system for providing a bone level percentage measurement of a dental image, the system comprising:
a processor, a memory configured to store instructions associated with the processor, the processor coupled to the memory; the processor may execute an instruction in any order;
wherein the processor includes:
image processing algorithms configured to:
receive the dental image and compensate for at least one of: a distorted image information, a missing image information, an obstructed image information;
process the dental image with a heuristic periapical bitewing algorithm to classify a dental image as one of: a periapical (PA) dental image, a bitewing (BW) dental image, a non-dental image;
wherein a non-dental image is replaced with a notification;
for the PA dental image:
process the PA dental image with at least one algorithm comprising: a machine learning (ML) cementoenamel junction (CEJ) algorithm, a ML first intersection of coronal alveolar bone and tooth algorithm, a ML apex of root algorithm;
identify a CEJ on the PA dental image with the ML CEJ algorithm;
identify a first intersection of coronal alveolar bone and tooth on the PA dental image with the ML first intersection of coronal alveolar bone and tooth algorithm;
identify an apex of root on the PA dental image with the ML apex of root algorithm;
measure the shortest contiguous distance on the PA dental image from the identified CEJ to the identified first intersection of coronal alveolar bone and tooth;
measure the shortest contiguous distance on the PA dental image from the identified CEJ to the identified apex of root;
calculate and derive:
the measured shortest contiguous distance on the PA dental image from the identified CEJ to the identified first intersection of coronal alveolar bone and tooth (numerator) divided by the measured shortest contiguous distance on the PA dental image from the identified CEJ to the identified apex of root (denominator) to produce a bone level percentage measurement;
associate the bone level percentage measurement with the PA dental image;
for the BW dental image:
process a bitewing (BW) dental image with at least one algorithm comprising: the ML CEJ algorithm, the ML first intersection of coronal alveolar bone and tooth algorithm;
identify a CEJ on the BW dental image with the ML CEJ algorithm;
identify a first intersection of coronal alveolar bone and tooth on the BW dental image with the ML first intersection of coronal alveolar bone and tooth algorithm;
calculate and derive:
measure the shortest contiguous distance on the BW dental image from the identified CEJ to the identified first intersection of coronal alveolar bone and tooth and process with an image aspect ratio algorithm to derive at least one of: a bone level percentage measurement, a measurement;
associate at least one of: a bone level percentage measurement, a measurement with a BW dental image.

2. The system of claim 1, wherein the processor is configured to calculate the bone level percentage measurement on a PA dental image from the identified CEJ, and the identified first intersection of coronal alveolar bone and tooth, and the identified apex of root that are all located on the same surface of the tooth;
wherein the processor is configured to calculate at least one of: a bone level percentage measurement, a measurement on a BW dental image from the identified CEJ, and the identified first intersection of coronal alveolar bone and tooth that are all located on the same surface of the tooth;
wherein the same surface of the tooth is one of: a mesial, a distal, a buccal, a lingual, a facial surface of the tooth.

3. The system of claim 1, further comprising a user interface for displaying at least one of: the identified CEJ, the identified first intersection of coronal alveolar bone and tooth, the identified apex of root, the shortest contiguous distance on the PA dental image from the identified CEJ to the identified first intersection of coronal alveolar bone and tooth, the shortest contiguous distance on the BW dental image from the identified CEJ to the identified first intersection of coronal alveolar bone and tooth, the shortest contiguous distance on the PA dental image from the identified CEJ to the identified apex of root, the bone level percentage measurement, the measurement, a patient data, the dental image.

4. The system of claim 3, wherein the patient data may be obtained from one or more of: a user, a provider, an e-commerce organization, a ML entity, a cloud based storage, an algorithm, a bioinformatics dataset, a business, an insurance dataset;
wherein the processor is configured to compensate for a missing component of the patient data;
wherein the patient data associated with a dental image is verified compliant with a regulatory policy.

5. The system of claim 1, wherein at least one of: the bone level percentage measurement, the measurement may be expressed as at least one of: a decimal, a quotient, a percentage, a ratio, a whole number;
wherein the processor is configured to compensate for a location variance of at least one of: the identified CEJ, the identified first intersection of coronal alveolar bone and tooth, the identified apex of root;
wherein the first intersection of alveolar bone and tooth may be an apex of an infrabony pocket.

6. Wherein the heuristic periapical bitewing algorithm is further configured to classify the dental image as at least one of: a panoramic dental image, a cephalometric dental image;
for the panoramic dental image:
process the panoramic dental image with at least one algorithm comprising: the ML CEJ algorithm, the ML first intersection of coronal alveolar bone and tooth algorithm, the ML apex of root algorithm;
identify a CEJ on the panoramic dental image with the ML CEJ algorithm;
identify a first intersection of coronal alveolar bone and tooth on the panoramic dental image with the ML first intersection of coronal alveolar bone and tooth algorithm;
identify an apex of root on the panoramic dental image with the ML apex of root algorithm;
measure the shortest contiguous distance on the panoramic dental image from the identified CEJ to the identified first intersection of coronal alveolar bone and tooth;
measure the shortest contiguous distance on the panoramic dental image from the identified CEJ to the identified apex of root;
calculate and derive:
the shortest contiguous distance on the panoramic dental image from the identified CEJ to the identified first intersection of coronal alveolar bone and tooth (numerator) divided by the shortest contiguous distance on the panoramic dental image from the identified CEJ to the identified apex of root (denominator) to produce a bone level percentage measurement;
associate the bone level percentage measurement with the panoramic dental image;
for the cephalometric dental image:
process the cephalometric dental image with at least one algorithm comprising: the ML CEJ algorithm, the ML first intersection of coronal alveolar bone and tooth algorithm, the ML apex of root algorithm;
identify a CEJ on the cephalometric dental image with the ML CEJ algorithm;
identify a first intersection of coronal alveolar bone and tooth on the cephalometric dental image with the ML first intersection of coronal alveolar bone and tooth algorithm;
identify an apex of root on a cephalometric dental image with the ML apex of root algorithm;
measure the shortest contiguous distance on the cephalometric dental image from the identified CEJ to the identified first intersection of coronal alveolar bone and tooth;
measure the shortest contiguous distance on the cephalometric dental image from the identified CEJ to the identified apex of root;
calculate and derive:
the shortest contiguous distance on the cephalometric dental image from the identified CEJ to the identified first intersection of coronal alveolar bone and tooth (numerator) divided by the shortest contiguous distance on the cephalometric dental image from the identified CEJ to the identified apex of root (denominator) to produce a bone level percentage measurement;
associate the bone level percentage measurement with the cephalometric dental image.

7. A system for providing an anatomic delineation percentage measurement of a dental image, the system comprising: a processing device configured to:
receive the dental image;
the processing device may execute an instruction in any order;
process the dental image using at least one machine learning (ML) algorithm comprising: a ML anatomy algorithm, a ML pathology algorithm to identify anatomic delineations in the dental image;
measure the distance on the dental image between a first identified anatomic delineation and a second identified anatomic delineation, and the distance on the dental image between a first identified anatomic delineation and a third identified anatomic delineation;
calculate the anatomic delineation percentage measurement by dividing the distance on the dental image between the first and second identified anatomic delineations (numerator) by the distance on the dental image between the first and third identified anatomic delineations (denominator);
associate the anatomic delineation percentage measurement with the dental image.

8. The system of claim 7, wherein the dental image is associated with a patient data.

9. The system of claim 8, wherein the patient data is associated with a dental image is verified compliant with a regulatory policy.

10. The system of claim 8, wherein the patient data may be obtained from one or more of: a user, a provider, an e-commerce organization, a ML entity, cloud based storage, an algorithm, a bioinformatics dataset, a business, an insurance dataset.

11. The system of claim 7, wherein the processing device calculates the anatomic delineation percentage measurements using a weighted average of distances between the identified anatomic delineations.

12. The system of claim 7, wherein the processing device applies image preprocessing techniques to the dental image before processing with ML algorithms.

13. The system of claim 7, wherein the processing device is configured to compensate for at least one of: a distorted information, a missing image, an obstructed information on the dental image.

14. The system of claim 7, wherein the processing device identifies at least one of: the first, the second, the third anatomic delineation(s) using a combination of ML anatomy and pathology algorithms.

15. The system of claim 7, further comprising a user interface for displaying the dental image associated with the anatomic delineation percentage measurement.

16. The system of claim 7, wherein the dental image is obtained from an image capture device.

17. A system for providing a bone level percentage measurement of a dental image, the system comprising:
a microprocessor, wherein said microprocessor may execute an instruction in any order is configured to:
receiving a dental image of a patient from a dental image provider;
compensating for at least one of: a distorted image information, a missing image information, an obstructed image information;
processing said dental image with a heuristic periapical bitewing algorithm to classify said dental image as one of: a periapical (PA) dental image, a bitewing (BW) dental image, a non-dental image;
wherein said non-dental image is replaced with a notification;
for said PA dental image:
processing said PA dental image with at least one algorithm comprising: a machine learning (ML) cementoenamel junction (CEJ) algorithm, a ML first intersection of coronal alveolar bone and tooth algorithm, a ML apex of root algorithm;
identifying a CEJ on said PA dental image with said ML CEJ algorithm to produce an identified CEJ;
identifying a first intersection of coronal alveolar bone and tooth on said PA dental image with said ML first intersection of coronal alveolar bone and tooth algorithm to produce an identified first intersection of coronal alveolar bone and tooth;
identifying an apex of root on said PA dental image with said ML apex of root algorithm to produce an identified apex of root;
measuring the shortest contiguous distance on said PA dental image from said identified CEJ to said identified first intersection of coronal alveolar bone and tooth;
measuring the shortest contiguous distance on said PA dental image from said identified CEJ to said identified apex of root;
deriving a percentage from:
said measuring the shortest contiguous distance on the PA dental image from said identified CEJ to said identified first intersection of coronal alveolar bone and tooth (numerator) divided by said measuring the shortest contiguous distance on the PA dental image from said identified CEJ to said identified apex of root (denominator) to produce a bone level percentage measurement;
querying and receiving a patient data and associate with said PA dental image of a patient;
associating said PA dental image and said patient data with at least one of: said identified CEJ, said identified first intersection of coronal alveolar bone and tooth, said identified apex of root, said shortest contiguous distance on said PA dental image from said identified CEJ to said identified first intersection of coronal alveolar bone and tooth, said shortest contiguous distance on said PA dental image from said identified CEJ to said identified apex of root, said bone level percentage measurement to produce a correlated dental information;
providing said correlated dental image information to a memory;
providing correlated dental information to one or more of: a user, a provider, an e-commerce organization, an insurance company, a bioinformatics organization, a business, a ML entity, a cloud based storage, an algorithm;
for said BW dental image:
processing said BW dental image with at least one algorithm comprising: said ML CEJ algorithm, said ML first intersection of coronal alveolar bone and tooth algorithm;
identifying a CEJ on said BW dental image with said ML CEJ algorithm to produce an identified CEJ;
identifying a first intersection of coronal alveolar bone and tooth on said BW dental image with said ML first intersection of coronal alveolar bone and tooth algorithm to produce an identified first intersection of coronal alveolar bone and tooth;
measuring the shortest contiguous distance on said BW dental image from said identified CEJ to said identified first intersection of coronal alveolar bone and tooth and process with an image aspect ratio algorithm to produce at least one of: a bone level percentage measurement, a measurement;
querying and receiving said patient data and associate with said BW dental image of said patient;
associating said BW dental image and said patient data with at least one of: said identified CEJ, said identified first intersection of coronal alveolar bone and tooth, said shortest contiguous distance on said BW dental image from said identified CEJ to said identified first intersection of coronal alveolar bone and tooth, said bone level percentage measurement, said measurement to produce a correlated dental information;
providing said correlated dental image information to a memory;
providing correlated dental information to one or more of: a user, a provider, an e-commerce organization, an insurance company, a bioinformatics organization, a business, a ML entity, a cloud based storage, an algorithm.

18. The system of claim 17, further comprising a user interface for displaying at least one of: said PA dental image, said BW dental image, said identified CEJ, said identified first intersection of coronal alveolar bone and tooth, said identified apex of root, said measuring the shortest contiguous distance on the PA dental image from said identified CEJ to said identified first intersection of coronal alveolar bone and tooth, said measuring the shortest contiguous distance on the PA dental image from said identified CEJ to said identified apex of root, said shortest contiguous distance on said BW dental image from said identified CEJ to said identified first intersection of coronal alveolar bone and tooth, said bone level percentage measurement, said measurement, said patient data, and said correlated dental information.

19. The system of claim 17, wherein said dental image provider obtains said dental image from at least one of: an image capture device, a data storage device;
wherein said microprocessor is configured to compensate for a missing component of said patient data;
wherein said processor may be configured to identify said identified CEJ at any location within the CEJ;
wherein said processor may be configured to identify said identified first intersection of coronal alveolar bone and tooth at any location on the alveolar bone;
wherein said processor may be configured to identify said identified apex of root at any location on the apex of root;
wherein said first intersection of alveolar bone and tooth may be an apex of infrabony pocket;
wherein the heuristic periapical bitewing algorithm is further configured to classify the dental image as at least one of: a panoramic dental image, a cephalometric dental image and continue processing.

20. The system of claim 17, wherein the microprocessor is configured to calculate said bone level percentage measurement on a PA dental image from said identified CEJ, and said identified first intersection of coronal alveolar bone and tooth, and said identified apex of root that are all located on the same surface of the tooth;
wherein the microprocessor is configured to calculate at least one of: said bone level percentage measurement, said measurement on a BW dental image from said identified CEJ, and said identified first intersection of coronal alveolar bone and tooth that are all located on the same surface of the tooth;
wherein the same surface of the tooth is one of: a mesial, a distal, a buccal, a lingual, a facial surface of the tooth.
